(19) United States
(12) Patent Application Publication
(10) Pub. No.: US 2024/0228467 A1
(43) Pub. Date: Jul. 11, 2024

(54) INDIRUBIN COMPOUNDS AND METHODS THEREOF

(71) Applicant: JAWAHARLAL NEHRU CENTRE FOR ADVANCED SCIENTIFIC RESEARCH, Jakkur, Bangalore (IN)

(72) Inventors: James Premdoss Clement CHELLIAH, Jakkur, Karnataka Bangalore (IN); Ravi MANJITHAYA, Jakkur, Karnataka Bangalore (IN); Sridhar RAJARAM, Jakkur, Karnataka Bangalore (IN); Vijaya VERMA, Jakkur, Karnataka Bangalore (IN); Kavita SHARMA, Jakkur, Karnataka Bangalore (IN); Suresh Santhi NATESAN, Jakkur, Karnataka Bangalore (IN)

(21) Appl. No.: 18/557,800

(22) PCT Filed: Apr. 29, 2022
(86) PCT No.: PCT/IN2022/050409

§ 371 Date: Oct. 27, 2023

(30) Foreign Application Priority Data
Apr. 29, 2021 (IN) 202141019766

(51) Int. Cl. C07D 403/06; A61K 31/404; C07D 403/14

(52) U.S. Cl. CPC C07D 403/06; A61K 31/404; C07D 403/14

(57) ABSTRACT

The present disclosure discloses a compound of Formula I and the process of preparation thereof. The present disclosure discloses indirubin compounds of Formula I which are potent inducers of autophagy. The present disclosure also discloses a pharmaceutical composition comprising compound of Formula I and methods thereof.

### FIELD OF THE INVENTION

The present disclosure relates to the field of heterocyclic compounds. The present disclosure relates to novel indirubin compounds and specifically relates to compounds of Formula I. The compounds of the present disclosure could further be used in combination with pharmaceutically acceptable carrier. The present disclosure also provides a process of preparation and pharmaceutical activity of the compounds of Formula I.

### BACKGROUND OF THE INVENTION

Everyday activities ranging from simplest walking to complex computation is regulated by coordinated brain activity. This in turn is regulated by synaptic connections in brain, which has remarkable property to undergo synaptic plasticity. The orchestrated activity of the brain requires several genes and proteins which are important for synaptic function. If any of these genes have mutation, then that might result in several neurological disorders such as Autism spectrum disorder (ASD) and Intellectual disability (ID). Current therapeutics to treat these disorders are mostly related to alleviating the symptoms rather than correct the phenotypes observed in neurodevelopmental disorder, especially after brain development. The most strategic therapies target the symptoms associated, such as seizures, hypotonia, sleeplessness, mania, euphoria, etc. Cholesterol-lowering statins, such as lovastatin or atorvastatin, has been proven to ameliorates deficits in global behaviour and cognitive behavior, however high dosage of atorvastatin for long-term could cause significant deficits in cognitive capabilities and necessary exploratory behavior. Commercially available lithium-based drugs or its derivatives are primarily used for the treatment of psychiatric disorders. However, chronic usage of lithium-based drugs results in several side effects due to Lithium toxicity including vomiting, tremor, or fatigue, enuresis, or irritability. The increased dosage might also cause acute renal failure, seizures, altered mental status, confusion, and coma.

Latest research efforts are done in screening of small molecules towards identifying and developing new therapeutics for neurodevelopmental disorders. Of the various strategies to develop such drugs, the one pathway that is explored recently is by inhibiting the GSK-3β receptor. Various pre-clinical studies have mostly targeted RAS-ERK signaling or more recently GSK-3β as therapeutics for ASD/ID. Braat S. and Kooy R. F. in 2015 (PMID: 26050032) summarized GABA receptor as a potential therapeutic target for neurodevelopmental disorders such as ASD/ID.

KR20170086547A discloses heteroaryl compounds as inhibitors of protein kinases and their pharmaceutical composition for treating various disorders.

U.S. Pat. No. 7,045,519B2 discloses pyrimidine or pyridine-based compounds, compositions, and methods of inhibiting GSK 3 and of treatment of GSK3 mediated disorders. However, there are various hindrances in designing these molecules as drugs, due to their poor solubility, poor bioavailability and in their dosage efficacies.

In view of the above discussion, it can be seen that there are still wide requirements in developing drug molecules especially small molecules which can act as GSK-3β inhibitors and thereby exploring the scope in treating various neurodevelopmental disorders.

### SUMMARY OF THE INVENTION

In one aspect of the present disclosure there is provided a compound of Formula I,

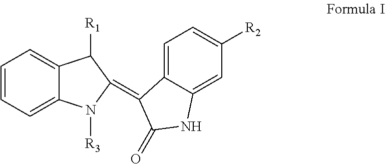

and its polymorphs, stereoisomers, prodrugs, solvates, co-crystals, intermediates, pharmaceutically acceptable salts, and metabolites thereof,
wherein R₁ is ═N—O-A,
wherein A is selected from hydrogen, C₁₋₁₀ alkyl, C₂₋₁₀ alkenyl, C₂₋₁₀ alkynyl, C₆₋₁₄ aryl, or —((CH₂)ₘ—O—)ₙH, and wherein C₁₋₁₀ alkyl, C₂₋₁₀ alkenyl, C₂₋₁₀ alkynyl, or C₆₋₁₄ aryl is optionally substituted with one or more groups selected from C₁₋₁₀ alkyl, C₂₋₁₀ alkenyl, C₂₋₁₀ alkynyl, C₆₋₁₄ aryl, —B, or —COO—C₁₋₁₀ alkyl-D, wherein —COO—C₁₋₁₀ alkyl-D is optionally substituted with —OCO—C₁₋₁₀ alkyl-E, or —COO—C₁₋₁₀ alkyl-E, wherein —OCO—C₁₋₁₀ alkyl-E, or —COO—C₁₋₁₀ alkyl-E is optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E and F are independently selected from C₁₋₁₀ alkyl, C₂₋₁₀ alkenyl, C₂₋₁₀ alkynyl, C₆₋₁₄ aryl, —COO—CH₂—C₆₋₁₄ aryl, C₁₋₂₀ heterocyclyl or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted;
R₂ is selected from hydrogen, halogen, C₁₋₁₀ alkyl, C₂₋₁₀ alkenyl, C₂₋₁₀ alkynyl, C₆₋₁₄ aryl, nitro, amine, or amide, wherein C₁₋₁₀ alkyl, C₂₋₁₀ alkenyl, or C₆₋₁₄ aryl is optionally substituted with one or more C₆₋₁₄ aryl, or halogen;
R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH; m is 1 to 3; and n is 1 to 10.

In another aspect of the present disclosure, there is provided a compound of Formula Ia

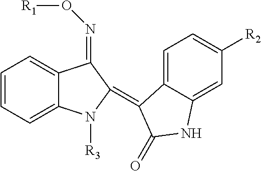

and its polymorphs, stereoisomers, prodrugs, solvates, co-crystals, intermediates, pharmaceutically acceptable salts, and metabolites thereof, wherein R₁ is selected from hydrogen, C₁₋₁₀ alkyl, or —((CH₂)ₘ—O—)ₙH, and C₁₋₁₀ alkyl is optionally substituted with one or more groups selected from C₆₋₁₄ aryl, —B, or —COO—C₁₋₁₀ alkyl-D, wherein —COO—C₁₋₁₀ alkyl-D is optionally substituted with —OCO—C₁₋₁₀ alkyl-E, or —COO—C₁₋₁₀ alkyl-E, wherein —OCO—C₁₋₁₀ alkyl-E, or —COO—C₁₋₁₀ alkyl-E is optionally substituted with —F, wherein B, D, E, and F are independently selected from

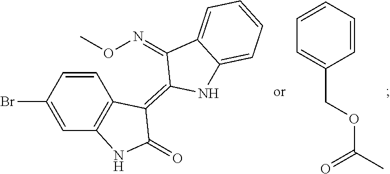

R₂ is selected from halogen, C₁₋₁₀ haloalkyl, C₂₋₁₀ alkenyl, or nitro, wherein C₂₋₁₀ alkenyl is optionally substituted with C₆₋₁₄ aryl; R₃ is selected from hydrogen, or —((CH₂)ₘ—O—)ₙH; m is 2; and n is 1 to 5.

In yet another aspect of the present disclosure, there is provided a process for preparing the compound of Formula I, wherein the process comprising: reacting a compound of Formula II with a compound of Formula III in the presence of a solvent and a base to obtain the compound of Formula I,

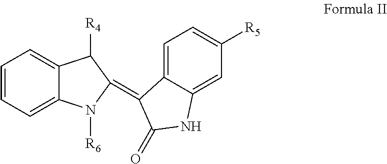

wherein R₄ is selected from hydrogen, ═N—O—H or ═O; R₅ is halogen, or nitro; R₆ is selected from hydrogen, C₁₋₁₀ alkyl, C₂₋₁₀ alkenyl, C₂₋₁₀ alkynyl, or C₆₋₁₄ aryl, wherein C₁₋₁₀ alkyl, C₂₋₁₀ alkenyl, C₂₋₁₀ alkynyl substituted with −(O—(CH₂)ₘ—)ₙ—OH; m is 1 to 3 and n is 1 to 10.

In one another aspect of the present disclosure, there is provided a pharmaceutical composition comprising a compound of Formula I or a pharmaceutically acceptable salt thereof together with a pharmaceutically acceptable carrier, optionally in combination with one or more other pharmaceutical compositions.

In further aspect of the present disclosure, there is provided a method for the treatment and/or prevention of various diseases, including intellectual disability (ID) or autism spectrum disorder comprising administering to a subject suffering from functional, behavioral, neurodegenerative disorders or intellectual disability (ID) or autism spectrum disorder a therapeutically effective amount of the compound to a subject in need thereof.

In another aspect of the present disclosure, there is provided a method for the treatment and/or prevention of various diseases, including intellectual disability (ID) or autism spectrum disorder, comprising administering to a subject suffering functional, behavioral, neurodegenerative disorders or intellectual disability (ID) or autism spectrum disorder a therapeutically effective amount of the compound or the pharmaceutical composition, with other clinically relevant agents to a subject in need thereof.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF THE ACCOMPANYING DRAWINGS

The following drawings form part of the present specification and are included to further illustrate aspects of the present disclosure. The disclosure may be better understood by reference to the drawings in combination with the detailed description of the specific embodiments presented herein.

FIG. 1 depicts the autophagic flux of the compounds of Formula I, in accordance with an implementation of the present disclosure.

### DESCRIPTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or steps.

The term “including” is used to mean “including but not limited to”, “including” and “including but not limited to” are used interchangeably.

In the structural formulae given herein and throughout the present disclosure, the following terms have been indicated meaning, unless specifically stated otherwise.

Furthermore, the compound of Formula (I) can be its derivatives, analogs, tautomeric forms, stereoisomer's, diastereomers, geometrical isomers, polymorphs, solvates, intermediates, metabolites, prodrugs or pharmaceutically acceptable salts and compositions.

The compounds described herein may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form, and mixtures thereof. Accordingly, the chemical structures depicted herein encompass all possible tautomeric forms of the illustrated or identified compounds. It is also understood that some isomeric form such as diastereomers, enantiomers and geometrical isomers can be separated by physical and/or chemical methods and by those skilled in the art. Pharmaceutically acceptable solvates may be hydrates or comprising of other solvents of crystallization such as alcohols, ether, and the like.

The term “solvate”, as used herein, refers to a crystal lattice which contains solvent.

The term “hydrate” refers to a more specific form of solvate, wherein the solvent is water.

The term “intermediate” refers to the compounds with same core structure of the compounds of the Formula I varying at specific allowed positions (for example alkyl chains). The term “metabolites” refers to end product of metabolism and which possess the functions of the compounds of Formula I. Metabolites are compounds obtained under physiological condition and would result in the compounds of Formula I.

As used herein, the term “substituted” is contemplated to include all permissible substituents of organic compounds. In a broad aspect, the permissible substituents include acyclic and cyclic, branched, and unbranched, carbocyclic, and heterocyclic, aromatic, and nonaromatic substituents of organic compounds. Illustrative substituents, for example, include those described hereinabove. The permissible substituents can be one or more and the same or different for appropriate organic compounds. For purposes of this disclosure, the heteroatoms such as nitrogen may have hydrogen substituents, and/or any permissible substituents of organic compounds described herein which satisfy the valences of the heteroatoms.

The term “alkyl” refers to straight or branched aliphatic hydrocarbon groups having 1 to 10 carbon atoms, which are attached to the rest of the molecule by a single atom, which may be optionally substituted by one or more substituents. Preferred alkyl groups include, without limitation, methyl, ethyl, n-propyl, isopropyl, butyl, isobutyl, t-butyl, pentyl, hexyl, heptyl, octyl and the like. Alkyl group may be optionally substituted. The term COO-alkyl refers to —COO— or —OCO— or —OOC— group linked to alkyl, wherein COO-alkyl may be optionally substituted.

The term “alkenyl” refers to straight or branched aliphatic hydrocarbon groups having 2 to 10 carbon atoms with at least one carbon-carbon double bond, which are attached to the rest of the molecule by a single atom, which may be optionally substituted by one or more substituents. Preferred alkenyl groups include, without limitation, ethenyl, propenyl, butenyl, 1-methyl-2-buten-1-yl, and the like.

The term “alkynyl” refers to straight or branched aliphatic hydrocarbon groups having 2 to 10 carbon atoms with at least one carbon-carbon triple bond, which are attached to the rest of the molecule by a single atom, which may be optionally substituted by one or more substituents. Preferred alkyl groups include, without limitation, ethynyl, 2-propynyl (propargyl), 1-propynyl, and the like.

The term “aryl” refers to aromatic radicals having 6 to 10 carbon atoms, which may be optionally substituted by one or more substituents. Preferred aryl groups include, without limitation, phenyl, naphthyl, and the like.

The term “heterocyclyl” refers to a heterocyclic ring radical which may be optionally substituted by one or more substituents. The heterocyclyl ring radical may be attached to the main structure at any heteroatom or carbon atom that results in the creation of a stable structure.

Furthermore, the term “heterocyclyl” refers to a stable 1 to 20 membered rings radical, which consists of carbon atoms and from one to five heteroatoms selected from nitrogen, phosphorus, oxygen, and sulfur. For purpose of this invention the heterocyclic ring radical may be monocyclic, bicyclic, or tricyclic ring systems, and the nitrogen, phosphorus, carbon, or sulfur atoms in the heterocyclic ring radical may be optionally oxidized to various oxidation states. In addition, the nitrogen atom may be optionally quarternized; and the ring radical may be partially or fully saturated. The term —O—N—C₁₋₂₀ heterocyclyl refers to a group comprising oxygen followed by nitrogen linked to heterocyclyl group, wherein the linkage between oxygen, nitrogen and heterocyclyl shall be single bond, or double bond based on the valencies and further may be balanced in the presence of hydrogen.

The term “halogen” or “halo” refers to the halogen atoms such as bromine, chlorine, fluorine, or iodine. In the present disclosure, the preferable halogen is bromine.

The compounds described herein can also be prepared in any solid or liquid physical form, for example, the compound can be in a crystalline form, in amorphous form and have any particle size. Furthermore, the compound particles may be micronized or nanosized, or may be agglomerated, particulate granules, powders, oils, oily suspensions, or any other form of solid or liquid physical forms.

The compounds described herein may also exhibit polymorphism. This invention further includes different polymorphs of the compounds of the present invention. The term polymorph refers to a particular crystalline state of a substance, having particular physical properties such as X-ray diffraction, IR spectra, melting point, and the like.

The phrase “pharmaceutically acceptable” refers to compounds or compositions that are physiologically tolerable and do not typically produce allergic or similar untoward reaction, including but not limited to gastric upset or dizziness when administered to subjects.

Pharmaceutically acceptable salts forming part of this invention include salts derived from inorganic bases such as like Li, Na, K, Ca, Mg, Fe, Cu, Zn and Mn and ammonium, substituted ammonium salts, aluminum salts and the like; salts of organic bases such as N, N′-diacetylethylenediamine, glucamine, triethylamine, choline, dicyclohexylamine, benzylamine, trialkylamine, thiamine, guanidine, diethanolamine, α-phenylethylamine, piperidine, morpholine, pyridine. hydroxyethylpyrrolidine, hydroxyethylpiperidine and the like, salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, fumarates, citrates, succinates, palmoates, methanesulphonates, tosylates, benzoates, salicylates, hydroxynaphthoates, benzenesulfonates, ascorbates, glycerophosphates, ketoglutarates and the like.

Compounds disclosed herein may exist as single stereoisomers, racemates and or mixtures of enantiomers and/or diastereomers. All such single stereoisomers, racemates and mixtures thereof are intended to be within the scope of the subject matter described.

It is understood that included in the family of compounds of Formula (I) are isomeric forms including diastereomers, enantiomers, tautomers, and geometrical isomers in “E” or “Z” configurational isomer or a mixture of ‘E’ and ‘Z’ isomers. It is also understood that some isomeric form such as diastereomers, enantiomers and geometrical isomers can be separated by physical and/or chemical methods and by those skilled in the art.

Compounds disclosed herein include isotopes of hydrogen, carbon, oxygen, fluorine, chlorine, iodine and sulfur which can be incorporated into the compounds, such as not limited to ²H (D), ³H (T), c ¹¹C, ¹³C, ¹⁴C, ¹⁵N, ¹⁸F, ³⁵S, ³⁶Cl and ¹²⁵I. Compounds of this invention where in atoms were isotopically labeled for example radioisotopes such as ³H, ¹³C, ¹⁴C, and the like can be used in metabolic studies, kinetic studies and imaging techniques such as positron emission tomography used in understanding the tissue distribution of the drugs. Compounds of the invention where hydrogen is replaced with deuterium may improve the metabolic stability and pharmacokinetics properties of the drug such as in vivo half-life. Compounds of the invention where isotopically labeled ¹⁸F can be useful as PET imaging studies.

The term “polymorphs” refers to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice.

The term “prodrugs” refers to the precursor of the compound of Formula (I), which on administration undergoes chemical conversion by metabolic processes before becoming active pharmacological substances. In general, such prodrugs will be functional derivatives of a compound of the invention, which are readily convertible in vivo into a compound of the invention.

The term “co-crystals” refers to two or molecules which are linked together by hydrogen bonding to form a crystal structure. In general, the term co-crystals refer to the compounds of Formula I in the crystalline form along with another molecule which may crystal co-formers.

The term “autophagy” refers to a method or a process by which the body removes the damaged cells or dysfunctional components. Autophagy refers to a natural mechanism which clears intracellular superfluous or damaged macromolecules or organelles via the lysosome degradation pathway. Autophagy helps in regeneration of cells and cell organelles which play critical role in development, survival, and differentiation of the cells. Autophagy serves cell functions, enabling the breakdown and recycling of cellular materials, and helps balance energy demands during periods of stress. Autophagy also helps to prevent and under certain conditions, promote tumor progression. The abnormal accumulation of autophagic cells are associated with multiple neurodegenerative conditions/diseases and other diseases. The term “potent inducer of autophagy” refers to compounds of Formula I which can induce autophagy in the cells.

The term “GSK-3β receptor” refers to glycogen synthase kinase 3 receptor which responds particularly to the GSK-3β gene.

The term “GSK-3β inhibitor” refers to the compounds of Formula I which can inhibit the receptor GSK-3β and aids to control or prevent diseases/conditions induced by GSK-3β receptor. The diseases/conditions include but not limited to neurological/neurodegenerative disorders such as Alzhemier's, Parkinson's, Amyotrophic Lateral Sclerosis Ataxia, Stroke, Migraine, Muscular Dystrophy, Multiple Sclerosis, Bell's Palsy, Epilepsy and Seizures, Guillain-Barré Syndrome, bipolar disorders, and other diseases such as diabetes mellitus, cancer and so on.

Unless defined otherwise, all technical and scientific terms used herein have the same meaning as commonly understood to one of ordinary skill in the art to which this disclosure belongs. Although methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally equivalent products, compositions, and methods are clearly within the scope of the disclosure, as described herein.

As discussed in the background, GSK 3β inhibitors help in treating neurodevelopmental disorders. GSK3β associated with neurodevelopmental disorders pathogenesis by modulating 2 processes namely (i) β-amyloid buildup and (ii) formation of neurofibrillary tangles. It can be demonstrated at cellular level experiments that inhibiting GSK3β would ablate the expression of SNCA gene (alpha-synuclein) suggesting its role in synucleopathies. Synucleopathies are due to the abnormal accumulation of aggregates of alpha-synuclein (SNCA) protein in neurons, nerve fibres or glial cells. The accumulation of these aggregates disrupts proteostasis machineries such as chaperones, proteasome or autophagy leading to neuronal degeneration. Neuronal loss of autophagy function leads to aggregate formation and subsequent neurodegeneration suggesting the importance of autophagy in preventing aggregate buildup. Regulating autophagy can eliminate these protein aggregates and restore cellular homeostasis. Therefore, the present disclosure explores the possibility of small molecules towards inducing autophagy and acting as inhibitor for GSK 3β receptor. These small molecules are drug-like compounds that clear such protein aggregates (aggrephagy) and restore cell viability. A phenotypic-based small molecules are identified for its ability to clear SNCA aggregates and restore cellular homeostasis. Also, these compounds of Formula I have improved solubility in comparison to 6-BIO. Accordingly, the present disclosure provides the compounds of Formula I which are small molecules that are drug like compounds and can act as potent inducer of autophagy and therefore are therapeutically efficacious in treating neurodevelopmental disorders.

In an implementation of the present disclosure there is provided a compound of Formula I

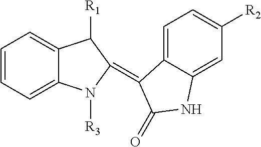

and its polymorphs, stereoisomers, prodrugs, solvates, co-crystals, intermediates, pharmaceutically acceptable salts, and metabolites thereof, wherein R₁ is ═N—O—A, wherein A is selected from hydrogen, C₁₋₁₀ alkyl, C₂₋₁₀ alkenyl, C₂₋₁₀ alkynyl, C₆₋₁₄ aryl, —((CH₂)ₘ—O—)ₙH, and wherein C₁₋₁₀ alkyl, C₂₋₁₀ alkenyl, C₂₋₁₀ alkynyl, or C₆₋₁₄ aryl is optionally substituted with one or more groups selected from C₁₋₁₀ alkyl, C₂₋₁₀ alkenyl, C₂₋₁₀ alkynyl, C₆₋₁₄ aryl, —B, —COO—C₁₋₁₀ alkyl-D, wherein —COO—C₁₋₁₀ alkyl-D is optionally substituted with —OCO—C₁₋₁₀ alkyl-E, or —COO—C₁₋₁₀ alkyl-E, wherein —OCO—C₁₋₁₀ alkyl-E, or —COO—C₁₋₁₀ alkyl-E is optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₁₀ alkyl, C₂₋₁₀ alkenyl, C₂₋₁₀ alkynyl, C₆₋₁₄ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is further optionally substituted; R₂ is selected from hydrogen, halogen, C₁₋₁₀ alkyl, C₂₋₁₀ alkenyl, C₂₋₁₀ alkynyl, C₆₋₁₄ aryl, nitro, amine, or amide, wherein C₁₋₁₀ alkyl, C₂₋₁₀ alkenyl, or C₆₋₁₄ aryl is optionally substituted with one or more C₆₋₁₄ aryl, or halogen; R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH; m is 1 to 3; and n is 1 to 10.

In an implementation of the present disclosure, there is provided a compound of Formula I as disclosed herein, wherein R₁ is ═N—O—A, wherein A is selected from hydrogen, C₁₋₈ alkyl, C₂₋₆ alkenyl, C₂₋₆ alkynyl, C₆₋₁₀ aryl, or —((CH₂)ₘ—O—)ₙH, and wherein C₁₋₈ alkyl, C₂₋₆ alkenyl, C₂₋₆ alkynyl, or C₆₋₁₀ aryl is optionally substituted with one or more groups selected from C₁₋₈ alkyl, C₂₋₆ alkenyl, C₂₋₆ alkynyl, C₆₋₁₀ aryl, —B, or —COO—C₁₋₈ alkyl-D, wherein —COO—C₁₋₈ alkyl-D is optionally substituted with —OCO—C₁₋₁₀ alkyl-E optionally substituted with —F, wherein B is selected from —COO—CH₂-C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E and F are independently selected from C₁₋₈ alkyl, C₂₋₆ alkenyl, C₆₋₁₄ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, or oxygen and is further optionally substituted; R₂ is selected from hydrogen, halogen, C₁₋₁₀ alkyl, C₂₋₁₀ alkenyl, C₂₋₁₀ alkynyl, C₆₋₁₄ aryl, or nitro, wherein C₁₋₁₀ alkyl, C₂₋₁₀ alkenyl, or C₆₋₁₄ aryl is optionally substituted with one or more C₆₋₁₄ aryl, or halogen; R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH; m is 1 to 3; and n is 1 to 10.

In an implementation of the present disclosure there is provided a compound of Formula I and its polymorphs, stereoisomers, prodrugs, solvates, co-crystals, intermediates, pharmaceutically acceptable salts, and metabolites thereof, wherein R₁ is ═N—O-A, wherein A is selected from hydrogen, C₁₋₆ alkyl, C₂₋₆ alkenyl, C₂₋₆ alkynyl, C₆₋₁₀ aryl, or —((CH₂)ₘ—O—)ₙH, and wherein C₁₋₆ alkyl, C₂₋₆ alkenyl, C₂₋₆ alkynyl, or C₆₋₁₀ aryl is optionally substituted with one or more groups selected from C₁₋₆ alkyl, C₂₋₆ alkenyl, C₂₋₆ alkynyl, C₆₋₁₀ aryl, —B, or —COO—C₁₋₅ alkyl-D, wherein —COO—C₁₋₅ alkyl-D is optionally substituted with —OCO—C₁₋₅ alkyl-E optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₆ alkyl, C₂₋₆ alkenyl, C₂₋₆ alkynyl, C₆₋₁₀ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; R₂ is selected from hydrogen, halogen, C₁₋₆ alkyl, C₂₋₆ alkenyl, C₂₋₆ alkynyl, C₆₋₁₀ aryl, nitro, amine, or amide, wherein C₁₋₆ alkyl, C₂₋₆ alkenyl, or C₆₋₁₀ aryl is optionally substituted with one or more C₆₋₁₀ aryl, or halogen; R₃ is selected from hydrogen, halogen, C₁₋₆ alkyl, C₂₋₆ alkenyl, C₂₋₆ alkynyl, C₆₋₁₀ aryl, or —((CH₂)ₘ—O—)ₙH; m is 1 to 3; and n is 1 to 10.

In an implementation of the present disclosure there is provided a compound of Formula I and its polymorphs, stereoisomers, prodrugs, solvates, co-crystals, intermediates, pharmaceutically acceptable salts, and metabolites thereof, wherein R₁ is ═N—O-A, wherein A is selected from hydrogen, C₁₋₆ alkyl, C₂₋₆ alkenyl, C₂₋₆ alkynyl, C₆₋₁₀ aryl, —((CH₂)ₘ—O—)ₙH, and wherein C₁₋₆ alkyl, C₂₋₆ alkenyl, C₂₋₆ alkynyl, or C₆₋₁₀ aryl is optionally substituted with one or more groups selected from C₁₋₆ alkyl, C₂₋₆ alkenyl, C₂₋₆ alkynyl, C₆₋₁₀ aryl, —B, or —COO—C₁₋₅ alkyl-D, wherein —COO—C₁₋₅ alkyl-D is optionally substituted with —OCO—C₁₋₅ alkyl-E optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₆ alkyl, C₂₋₆ alkenyl, C₂₋₆ alkynyl, C₆₋₁₀ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; R₂ is selected from hydrogen, halogen, C₁₋₆ alkyl, C₂₋₆ alkenyl, C₂₋₆ alkynyl, C₆₋₁₀ aryl, nitro, amine, or amide, wherein C₁₋₆ alkyl, C₂₋₆ alkenyl, or C₆₋₁₀ aryl is optionally substituted with one or more C₆₋₁₀ aryl, or halogen; R₃ is selected from hydrogen, halogen, C₁₋₆ alkyl, C₂₋₆ alkenyl, C₂₋₆ alkynyl, C₆₋₁₀ aryl, or —((CH₂)ₘ—O—)ₙH; m is 1 to 2; and n is 1 to 6.

In an implementation of the present disclosure there is provided a compound of Formula I and its polymorphs, stereoisomers, prodrugs, solvates, co-crystals, intermediates, pharmaceutically acceptable salts, and metabolites thereof, wherein R₁ is ═N—O-A, wherein A is selected from hydrogen, C₁₋₆ alkyl, C₂₋₆ alkenyl, C₂₋₆ alkynyl, C₆₋₁₀ aryl, —((CH₂)ₘ—O—)ₙH, and wherein C₁₋₆ alkyl, C₂₋₆ alkenyl, C₂₋₆ alkynyl, or C₆₋₁₀ aryl is optionally substituted with one or more groups selected from C₁₋₆ alkyl, C₂₋₆ alkenyl, C₂₋₆ alkynyl, C₆₋₁₀ aryl, —B, or —COO—C₁₋₅ alkyl-D, wherein —COO—C₁₋₅ alkyl-D is optionally substituted with —OCO—C₁₋₅ alkyl-E optionally substituted with —F, wherein B is selected from —COO—CH₂—C₆₋₁₄ aryl, C₁₋₂₀ heterocyclyl or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₆ alkyl, C₂₋₆ alkenyl, C₂₋₆ alkynyl, C₆₋₁₀ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally substituted with one or more groups selected from oxo, halogen, C₁₋₆ alkyl, C₂₋₆ alkenyl, C₂₋₆ alkynyl, C₆₋₁₀ aryl, or C₁₋₂₀ heterocyclyl; R₂ is selected from hydrogen, halogen, C₁₋₆ alkyl, C₂₋₆ alkenyl, C₂₋₆ alkynyl, C₆₋₁₀ aryl, nitro, amine, or amide, wherein C₁₋₆ alkyl, C₂₋₆ alkenyl, or C₆₋₁₀ aryl is optionally substituted with one or more C₆₋₁₀ aryl, or halogen; R₃ is selected from hydrogen, halogen, C₁₋₆ alkyl, C₂₋₆ alkenyl, C₂₋₆ alkynyl, C₆₋₁₀ aryl, or —((CH₂)ₘ—O—)ₙH; m is 1 to 2; and n is 1 to 6.

In an implementation of the present disclosure, there is provided a compound of Formula I as disclosed herein, wherein B, D, E, and F are independently selected from

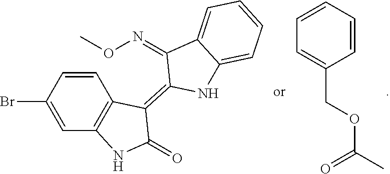

In an implementation of the present disclosure, there is provided a compound of Formula I as disclosed herein, wherein m is 2 and n is 1 to 5.

In an implementation of the present disclosure, there is provided a compound of Formula I

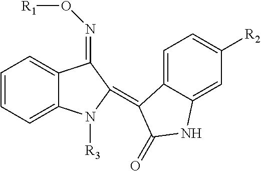

and its polymorphs, stereoisomers, prodrugs, solvates, co-crystals, intermediates, pharmaceutically acceptable salts, and metabolites thereof, wherein R₁ is selected from hydrogen, C₁₋₁₀ alkyl, —((CH₂)ₘ—O—)ₙH, and C₁₋₁₀ alkyl is optionally substituted with one or more groups selected from C₆₋₁₄ aryl, —B, or —COO—C₁₋₁₀ alkyl-D, wherein —COO—C₁₋₁₀ alkyl-D is optionally substituted with —OCO—C₁₋₁₀ alkyl-E, or —COO—C₁₋₁₀ alkyl-E, wherein —OCO—C₁₋₁₀ alkyl-E, or —COO—C₁₋₁₀ alkyl-E is optionally substituted with —F, wherein B, D, E, and F are independently selected from

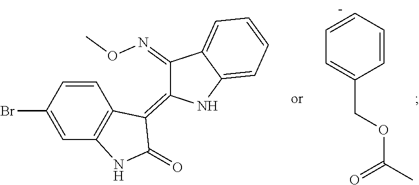 [SMILES: CO/N=C1\C(=C2\C(=O)Nc3cc(Br)ccc32)Nc2ccccc21 ; CC(=O)OCc1cc[c-]cc1]

R₂ is selected from halogen, C₁₋₁₀ haloalkyl, C₂₋₁₀ alkenyl, or nitro, wherein C₂₋₁₀ alkenyl is optionally substituted with C₆₋₁₄ aryl; R₃ is selected from hydrogen, or —((CH₂)ₘ—O—)ₙH; m is 2; and n is 1 to 5.

In an implementation of the present disclosure, there is provided a compound of Formula I as disclosed herein, wherein the compound of Formula I is selected from
a. (2Z,3E)-6′-bromo-3-(hydroxyimino)-[2,3′-biindolinylidene]-2′-one lithium salt;
b. (2Z,3E)-3-((benzyloxy)imino)-6′-bromo-[2,3′-biindolinylidene]-2′-one;
c. (2Z,3E)-6′-bromo-3-((2-(2-hydroxyethoxy)ethoxy)imino)-[2,3′-biindolinylidene]-2′-one;
d. (2Z,3E)-6′-bromo-3-((2-(2-(2-hydroxyethoxy)ethoxy)ethoxy)imino)-[2,3′-biindolinylidene]-2′-onee.(2Z,3E)-6′-bromo-3-(((14-hydroxy-3,6,9,12-tetraoxatetradecyl)oxy)imino)-[2,3′-biindolinylidene]-2′-one;
f. (2Z,3E)-6′-bromo-1-(2-(2-hydroxyethoxy)ethyl)-3-(hydroxyimino)-[2,3′-biindolinylidene]-2′-one;
g. (2Z,3E)-6′-bromo-1-(2-(2-(2-hydroxyethoxy)ethoxy)ethyl)-3-(hydroxyimino)-[2,3′-biindolinylidene]-2′-one;
h. benzyl 3-(((E)-((Z)-6′-bromo-2′-oxo-[2,3′-biindolinylidene]-3-ylidene)amino)oxy)-2-((((Z)—((Z)-6′-bromo-2′-oxo-[2,3′-biindolinylidene]-3-ylidene)amino)oxy)methyl)-2-methylpropanoate;
i. 2-((benzyloxy)carbonyl)-2-methylpropane-1,3-diyl bis(3-((((2Z,3E)-6′-bromo-2′-oxo-[2,3′-biindolinylidene]-3-ylidene)amino)oxy)-2-(((((2Z,3E)-6′-bromo-2′-oxo-[2,3′-biindolinylidene]-3-ylidene)amino)oxy)methyl)-2-methylpropanoate)
j. (2Z,3E)-1-(2-(2-(2-hydroxyethoxy)ethoxy)ethyl)-3-(hydroxyimino)-6′-((E)-styryl)-[2,3′-biindolinylidene]-2′-one;
k. (2Z,3E)-6′-bromo-3-((2-(2-(2-hydroxyethoxy)ethoxy)ethoxy)imino)-1-(2-(2-(2-hydroxyethoxy)ethoxy)ethyl)-[2,3′-biindolinylidene]-2′-one;
l. (2Z,3E)-3-(hydroxyimino)-6′-nitro-[2,3′-biindolinylidene]-2′-one lithium salt;
m. (2Z,3E)-6′-bromo-3-((2-hydroxyethoxy)imino)-[2,3′-biindolinylidene]-2′-one;
n. (2Z,3E)-1-(2-hydroxyethyl)-3-(hydroxyimino)-6′-bromo-[2,3′-biindolinylidene]-2′-one lithium salt;
o. (2Z,3E)-3-(hydroxyimino)-6′-((E)-styryl)-[2,3′-biindolinylidene]-2′-one; and
p. (2Z,3E)-3-(hydroxyimino)-6′-nitro-[2,3′-biindolinylidene]-2′-one.

In an implementation of the present disclosure, there is provided the compound of Formula I as disclosed herein, wherein the compound of Formula I acts as potent inducer of autophagy.

In an implementation of the present disclosure, there is provided the compound of Formula I as disclosed herein, wherein the compound of Formula I inhibits GSK-3β receptor.

In an implementation of the present disclosure, there is provided an intermediate compound of Formula IA, the Formula IA selected from (Z)-6′-bromo-1-(2-(2-(2-hydroxyethoxy)ethoxy)ethyl)-[2,3′-biindolinylidene]-2′,3-dione and (Z)-6′-bromo-1-(2-(2-hydroxyethoxy)ethyl)-[2,3′-biindolinylidene]-2′,3-dione.

In an implementation of the present disclosure, there is provided a compound of Formula I and its pharmaceutically acceptable salts thereof, wherein the pharmaceutically acceptable salts are derived from inorganic bases such as Li, Na, K, Ca, Mg, Fe, Cu, Zn and Mn and ammonium, substituted ammonium salts, and aluminum salts.

In an implementation of the present disclosure, there is provided a process for preparing the compound of Formula I as disclosed herein, the process comprising: reacting a compound of Formula II with a compound of Formula III in the presence of a solvent and a base to obtain the compound of Formula I,

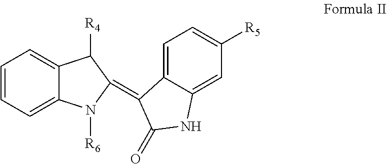

wherein R₄ is selected from hydrogen, ═N—O—H or ═O; R₅ is halogen or nitro; R₆ is selected from hydrogen, C₁₋₁₀ alkyl, C₂₋₁₀ alkenyl, C₂₋₁₀ alkynyl, or C₆₋₁₄ aryl wherein C₁₋₁₀ alkyl C₂₋₁₀ alkenyl, or C₂₋₁₀ alkynyl is optionally substituted with —(O—(CH₂)ₘ—)ₙ—OH; m is 1 to 3 and n is 1 to 10.

In an implementation of the present disclosure, there is provided the process for preparing the compound of Formula I as disclosed herein, wherein the compound of Formula III is selected from A-X, R₂—X, or R₃—X, wherein A is selected from hydrogen, C₁₋₁₀ alkyl, C₂₋₁₀ alkenyl, C₂₋₁₀ alkynyl, C₆₋₁₄ aryl, or —((CH₂)ₘ—O—)ₙH, and wherein C₁₋₁₀ alkyl, C₂₋₁₀ alkenyl, C₂₋₁₀ alkynyl, or C₆₋₁₄ aryl is optionally substituted with one or more groups selected from C₁₋₁₀ alkyl, C₂₋₁₀ alkenyl, C₂₋₁₀ alkynyl, C₆₋₁₄ aryl, —B, or —COO—C₁₋₁₀ alkyl-D, wherein —COO—C₁₋₁₀ alkyl-D is optionally substituted with —OCO—C₁₋₁₀ alkyl-E, or —COO—C₁₋₁₀ alkyl-E, wherein —OCO—C₁₋₁₀ alkyl-E, or —COO—C₁₋₁₀ alkyl-E is optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₁₀ alkyl, C₂₋₁₀ alkenyl, C₂₋₁₀ alkynyl, C₆₋₁₄ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur and is optionally further substituted; R₂ is selected from hydrogen, halogen, C₁₋₁₀ alkyl, C₂₋₁₀ alkenyl, C₂₋₁₀ alkynyl, C₆₋₁₄ aryl, nitro, amine, or amide, wherein C₁₋₁₀ alkyl, C₂₋₁₀ alkenyl, or C₆₋₁₄ aryl is optionally substituted with one or more C₆₋₁₄ aryl, or halogen; R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH; m is 1 to 3; n is 1 to 10; and X is selected from hydrogen, halogen, or tosyl.

In an implementation of the present disclosure, there is provide a process for preparing the compound of Formula I as disclosed herein, wherein B, D, E, and F are independently selected from

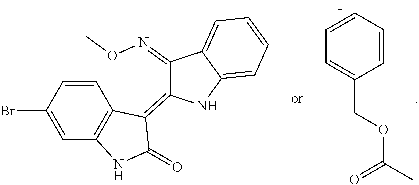 [SMILES: CO/N=C1\C(=C2\C(=O)Nc3cc(Br)ccc32)Nc2ccccc21 ; CC(=O)OCc1cc[c-]cc1]

In an implementation of the present disclosure, there is provide a process for preparing the compound of Formula I as disclosed herein, wherein the solvent is selected from tetrahydrofuran, pyridine, dimethyl furan, methanol, N,N′-dimethyl formamide, dichloromethane, or combinations thereof.

In an implementation of the present disclosure, there is provide a process for preparing the compound of Formula I as disclosed herein, wherein the base is selected from potassium carbonate, triethyl amine, sodium bicarbonate, pyridine, lithium hydroxide, sodium hydroxide, lithium hydroxide, or combinations thereof.

In an implementation of the present disclosure, there is provide a process for preparing the compound of Formula I as disclosed herein, wherein the process comprises a catalyst selected from palladium acetate, tri(o-tolyl)phosphine, or combinations thereof.

In an implementation of the present disclosure, there is provide a process for preparing the compound of Formula I as disclosed herein, the process comprising: reacting a compound of Formula II with a compound of Formula III selected from A-X, R₂—X, or R₃—X in the presence of a solvent selected from tetrahydrofuran, pyridine, dimethyl furan, methanol, N,N′-dimethyl formamide, dichloromethane, or combinations thereof and a base selected from potassium carbonate, triethyl amine, sodium bicarbonate, pyridine, lithium hydroxide, sodium hydroxide, lithium hydroxide, or combinations thereof to obtain the compound of Formula I, wherein A is selected from hydrogen, C₁₋₁₀ alkyl, C₂₋₁₀ alkenyl, C₂₋₁₀ alkynyl, C₆₋₁₄ aryl, or —((CH₂)ₘ—O—)ₙH, and wherein C₁₋₁₀ alkyl, C₂₋₁₀ alkenyl, C₂₋₁₀ alkynyl, or C₆₋₁₄ aryl is optionally substituted with one or more groups selected from C₁₋₁₀ alkyl, C₂₋₁₀ alkenyl, C₂₋₁₀ alkynyl, C₆₋₁₄ aryl, —B, or —COO—C₁₋₁₀ alkyl-D, wherein —COO—C₁₋₁₀ alkyl-D is optionally substituted with —COO—C₁₋₁₀ alkyl-E, or —OCO—C₁₋₁₀ alkyl-E, wherein —COO—C₁₋₁₀ alkyl-E, or —OCO—C₁₋₁₀ alkyl-E is optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₁₀ alkyl, C₂₋₁₀ alkenyl, C₂₋₁₀ alkynyl, C₆₋₁₄ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur and is further optionally substituted; R₂ is selected from hydrogen, halogen, C₁₋₁₀ alkyl, C₂₋₁₀ alkenyl, C₂₋₁₀ alkynyl, C₆₋₁₄ aryl, nitro, amine, or amide, wherein C₁₋₁₀ alkyl, C₂₋₁₀ alkenyl, or C₆₋₁₄ aryl is optionally substituted with one or more C₆₋₁₄ aryl, or halogen; R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH; X is selected from hydrogen, halogen, or tosyl; R₄ is selected from hydrogen, ═N—O—H, or ═O; R₅ is halogen, or nitro; R₆ is selected from hydrogen, C₁₋₁₀ alkyl, C₂₋₁₀ alkenyl, C₂₋₁₀ alkynyl, or C₆₋₁₄ aryl wherein C₁₋₁₀ alkyl C₂₋₁₀ alkenyl, or C₂₋₁₀ alkynyl is optionally substituted with —(O—(CH₂)ₘ—)ₙ—OH; m is 1 to 3 and n is 1 to 10.

In an implementation of the present disclosure, there is provided a pharmaceutical composition comprising a compound of Formula I or a pharmaceutically acceptable salt thereof as disclosed herein together with a pharmaceutically acceptable carrier, optionally in combination with one or more other pharmaceutical compositions.

In an implementation of the present disclosure, there is provided the pharmaceutical composition as disclosed herein, wherein the composition is in the form selected from the group consisting of a tablet, capsule, powder, syrup, solution, aerosol, and suspension.

In an implementation of the present disclosure, there is provided the pharmaceutical composition as disclosed herein, wherein the composition acts as potent inducer of autophagy.

In an implementation of the present disclosure, there is provided the pharmaceutical composition as disclosed herein, wherein the composition inhibits GSK-3β (glycogen synthase kinase-3) receptor.

In an implementation of the present disclosure, there is provided a method for the treatment and/or prevention of various diseases, including intellectual disability (ID) or autism spectrum disorder comprising administering to a subject suffering from functional, behavioral, neurodegenerative disorders or intellectual disability (ID) or autism spectrum disorder a therapeutically effective amount of the compound as disclosed herein to a subject in need thereof.

In an implementation of the present disclosure, there is provided a method for the treatment and/or prevention of various diseases, including intellectual disability (ID) or autism spectrum disorder, comprising administering to a subject suffering functional, behavioral, neurodegenerative disorders or intellectual disability (ID) or autism spectrum disorder a therapeutically effective amount of the compound of Formula I or the pharmaceutical composition as disclosed herein, with other clinically relevant agents to a subject in need thereof.

In an implementation of the present disclosure, there is provided use of the compounds of Formula I or the pharmaceutical composition as disclosed herein, for the treatment and/or prevention of various diseases including functional, behavioral, or neurodegenerative disorders, intellectual disability (ID) or autism spectrum disorder together with other clinically relevant cytotoxic agents or non-cytotoxic agents.

In an implementation of the present disclosure, there is provided use of the compounds of Formula I or the pharmaceutical composition as disclosed herein, for the treatment and/or prevention of various diseases including functional, behavioral or neurodegenerative disorders, intellectual disability (ID) or autism spectrum disorder, neurological/neurodegenerative disorders including alzheimer, parkinson's, amyotrophic lateral sclerosis ataxia, stroke, migraine, encephalitis, muscular dystrophy, multiple sclerosis, bell's palsy, epilepsy and seizures, guillain-barré syndrome, bipolar disorders and other diseases such as diabetes mellitus, cancer together with other clinically relevant cytotoxic agents or non-cytotoxic agents.

In an implementation of the present disclosure, there is provided a compound of Formula I for use in treating and/or preventing a neurodegenerative disorder or condition. In a related aspect, the invention provides for the use of a compound of Formula I for the manufacture of a medicament for treating and/or preventing a neurodegenerative disorder or condition.

In an implementation of the present disclosure, there is provided a compound which may be administered in combination therapy by combining the compound of Formula I with one or more relevant agents, and combination therapy includes the administration of the subject compounds in further combination with other biologically active ingredients (such as, but are not limited to, different antineoplastic agent) and non-drug therapies (such as, but are not limited to, surgery or radiation treatment). The compounds described herein can be used in combination with other pharmaceutically active compounds, preferably, which will enhance the effect of the compounds of the invention. The compounds can be administered simultaneously or sequentially to the other drug therapy.

Although the subject matter has been described in considerable detail with reference to certain examples and implementations thereof, other implementations are possible.

### EXAMPLES

The following examples provide the details about the synthesis, activities, and applications of the compounds of the present disclosure. It should be understood the following is representative only, and that the invention is not limited by the details set forth in these examples.

There is provided a process for preparing compounds of Formula I with all groups as defined earlier and the process comprised reacting a compound of Formula II with Formula III in the presence of a solvent and a base.

### Synthesis of (2Z,3E)-6′-bromo-3-(hydroxyimino)-[2,3′-biindolinylidene]-2′-one lithium salt (a)

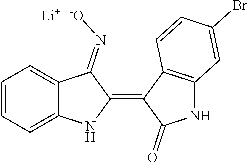

To a solution of 6-bromoindirubin oxime (0.364 mmol) in tetrahydrofuran (THF) (10 mL, HPLC grade), 2.5 mL of aqueous solution of lithium hydroxide (0.364 mmol) was added dropwise and the reaction mixture was stirred at room temperature for 10 min. The solvent was evaporated, washed with n-pentane (3×10 mL), and then dried on hi-vacuum to obtain quantitative yield of product. 1H NMR (DMSO-d₆, 600 MHZ): δ 12.42 (s, 1H), 10.28 (s, 1H), 8.60 (d, J=8.28 Hz, 1H), 8.37 (d, J=7.56 Hz, 1H), 7.25 (d, J=7.56 Hz, 1H), 7.08 (t, J=7.32 Hz, 1H), 6.90-6.94 (m, 3H); 13C NMR (DMSO-d₆, 100 MHZ): δ 170.2, 154.1, 152.2, 137.8, 136.1, 125.6, 125.3, 121.9, 121.6, 121.0, 120.8, 116.6, 112.6, 112.1, 109.9, 109.6, 88.4; HRMS for [C₁₆H₉⁷⁹BrLiN₃O₂+H]⁺: Calcd. 362.0116, Found: 362.008.

### Synthesis of (2Z,3E)-3-((benzyloxy)imino)-6′-bromo-[2,3′-biindolinylidene]-2′-one (b)

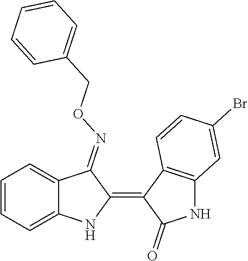 [SMILES: O=C1Nc2cc(Br)ccc2/C1=C1/Nc2ccccc2/C1=N\OCc1ccccc1]

In a 25 mL 2-neck round bottom flask 6-bromoindirubin (0.305 mmol) and O-benzylhydroxylamine hydrochloride (3.05 mmol) were added under argon. To this mixture dry pyridine (3 mL) was added and reaction mixture was refluxed at 120° C. for 1.5 h. The reaction mixture was cooled, diluted with water (100 mL), and then filtered. The residue was washed with more water and then dried. The crude product was purified using flash column chromatography (in 5% MeOH:DCM solution) to obtain compound b (73% yield). 1H NMR (DMSO-d₆, 600 MHZ): δ 11.66 (s, 1H), 10.88 (s, 1H), 8.31 (d, J=8.52 Hz, 1H), 8.17 (d, J=7.56 Hz, 1H), 7.58 (d, J=7.32 Hz, 2H), 7.42-7.46 (m, 4H), 7.37 (t, J=7.35 Hz, 1H), 7.03-7.06 (m, 2H), 6.990 (s, 1H), 5.63 (s, 2H); 13C NMR (DMSO-d₆, 100 MHz): δ 170.5, 151.7, 145.4, 144.3, 139.9, 137.1, 133.0, 128.6, 128.4, 128.3, 128.3, 124.7, 122.9, 121.7, 121.4, 118.2, 116.1, 112.0, 111.4, 99.1; HRMS for [C₂₃H₁₆⁷⁹BrN₃O₂+H]⁺: Calcd. 446.0504, Found: 446.0464.

### Synthesis of (2Z,3E)-6′-bromo-3-((2-(2-hydroxyethoxy)ethoxy)imino)-[2,3′-biindolinylidene]-2′-one (c)

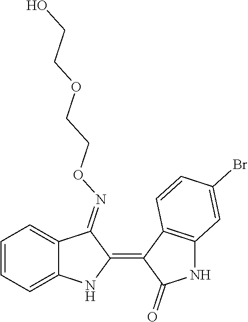 [SMILES: O=C1Nc2cc(Br)ccc2/C1=C1/Nc2ccccc2/C1=N\OCCOCCO]

In a 25 mL 2-neck round bottom flask compound a (0.276 mmol) was dissolved in dry DMF (5 mL) were added under argon. To this solution (2-(2-chloro)ethoxy)ethanol (5.52 mmol) was added drop wise and reaction mixture was stirred at room temperature for 2 h, followed by addition of 5.52 mmol of (2-(2-chloro)ethoxy)ethanol and stirred for another hour. The reaction mixture diluted with water (200 mL), filtered and the residue was then dried. The crude product was purified using flash column chromatography (in 1 to 2% MeOH:DCM solution) to obtain product. ¹H NMR (DMSO-d₆, 400 MHz): δ 11.69 (s, 1H), 10.09 (s, 1H), 8.52 (d, J=8.48 Hz, 1H), 8.15 (d, J=7.60 Hz, 1H), 7.42-7.46 (m, 2H), 7.14-7.15 (dd, J=8.44, 1.88 Hz, 1H), 7.02-7.07 (m, 2H), 4.71-4.73 (m, 2H), 4.60-4.63 (m, 1H), 3.91-3.93 (m, 2H), 3.49-3.54 (m, 4H); 13C NMR (DMSO-d₆, 100 MHz): 170.5, 151.4, 145.3, 144.5, 139.9, 132.9, 128.3, 124.6, 123.1, 121.7, 121.6, 118.2, 116.1, 111.9, 111.5, 98.9, 76.0, 72.4, 68.7, 60.2; HRMS for [C₂₀H₁₈⁷⁹BrN₃O₄+H]⁺: Calcd. 444.0559, Found: 444.0508.

### Synthesis of (2Z,3E)-6′-bromo-3-((2-(2-(2-hydroxyethoxy)ethoxy)ethoxy)imino)-[2,3′-biindolinylidene]-2′-one (d)

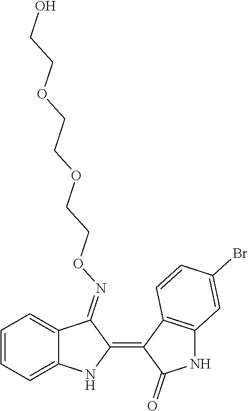 [SMILES: O=C1Nc2cc(Br)ccc2/C1=C1/Nc2ccccc2/C1=N\OCCOCCOCCO]

In a 25 mL 2-neck round bottom flask 6-bromoindirubin oxime (0.182 mmol) and potassium carbonate (0.456 mmol) was dissolved in dry DMF (5 mL) were added under argon. To this solution 2-[2-(2-chloroethoxy)ethoxy]ethanol (0.912 mmol) was added drop wise and reaction mixture was stirred at 45° C. for 24 h. The reaction mixture was cooled, diluted with water (100 mL), and extracted with ethyl acetate (3×25 mL). The organic layer was washed with water (3×30 mL) and then with brine. The organic layer was then dried over anhydrous Na₂SO₄ and filtered. The filtrate was concentrated and purified using flash column chromatography (using 0-3% MeOH: DCM) to obtain product (78% yield). 1H NMR (DMSO-d₆, 400 MHZ): δ 11.68 (s, 1H), 10.88 (s, 1H), 8.51 (d, J=8.32 Hz, 1H), 8.13 (d, J=7.40 Hz, 1H), 7.42 (bs, 2H), 7.14 (d, J=8.04, 1H), 7.02-7.05 (m, 2H), 4.71 (bs, 2H), 4.54 (t, J=4.60 Hz, 1H), 3.92 (bs, 2H), 3.39-3.62 (m, 8H); ¹³C NMR (DMSO-d₆, 100 MHz): δ 170.5, 151.3, 145.3, 144.5, 139.9, 132.9, 128.3, 124.6, 123.1, 121.7, 121.6, 118.2, 116.1, 111.9, 111.5, 99.0, 75.9, 72.3, 70.0, 69.7, 68.7, 60.1 HRMS for [C₂₂H₂₂⁷⁹BrN₃O₅+H]⁺: Calcd. 488.0821, Found: 488.0821.

### Synthesis of (2Z,3E)-6′-bromo-3-(((14-hydroxy-3,6,9,12-tetraoxatetradecyl)oxy)imino)-[2,3′-biindolinylidene]-2′-one (e)

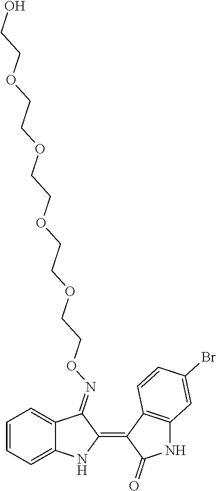 [SMILES: O=C1Nc2cc(Br)ccc2/C1=C1/Nc2ccccc2/C1=N\OCCOCCOCCOCCOCCO]

In a 25 mL 2-neck round bottom flask 6-bromoindirubin oxime (6-BIO) (0.136 mmol) and potassium carbonate (0.404 mmol) was dissolved in dry DMF (5 mL) were added under argon. To this solution compound 14-chloro-3,6,9,12-tetraoxatetradecan-1-ol (0.681 mmol) was added drop wise and reaction mixture was stirred at room temperature for 30 h. 6-BIO was not consumed so, potassium carbonate (0.404 mmol) was added and the reaction mixture was heated at 45° C. 6 h. The reaction mixture was cooled, diluted with water (100 mL), and extracted with ethyl acetate (2×50 mL). The organic layer was washed with water (3×30 mL) and then with brine. The organic layer was dried over anhydrous Na₂SO₄ and filtered. The filtrate was concentrated and purified using flash column chromatography (using 2-3% MeOH: DCM) to obtain product (32% yield). 1H NMR (DMSO-d₆, 400 MHz): δ 11.68 (s, 1H), 10.88 (s, 1H), 8.51 (d, J=8.32 Hz, 1H), 8.13 (d, J=7.40 Hz, 1H), 7.42 (bs, 2H), 7.14 (d, J=8.04, 1H), 7.02-7.05 (m, 2H), 4.71 (bs, 2H), 4.54 (t, J=4.60 Hz, 1H), 3.92 (bs, 2H), 3.39-3.62 (m, 8H); ¹³C NMR (DMSO-d₆, 100 MHZ): Sample was less, peaks did not come up properly; MALDI for [C₂₆H₃₀⁷⁹BrN₃O₇+H]⁺: Calcd. 576.1345, Found: 576.1313.

### Synthesis of (Z)-6′-bromo-1-(2-(2-hydroxyethoxy)ethyl)-[2,3′-biindolinylidene]-2′,3-dione (1)

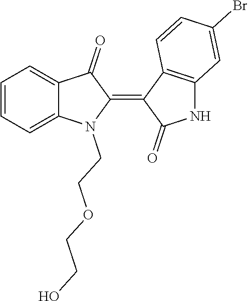 [SMILES: O=C1Nc2cc(Br)ccc2/C1=C1\C(=O)c2ccccc2N1CCOCCO]

In a 25 mL 2-neck round bottom flask 6-bromoindirubin (0.293 mmol) and potassium carbonate (0.351 mmol) were dissolved in dry DMF (5 mL) were added under argon. To this solution (2-(2-chloro)ethoxy)ethanol (1.46 mmol) was added drop wise and reaction mixture was stirred at room temperature for 1.5 h. The reaction mixture was then heated to 45° C. for 18 h. Potassium carbonate (0.351 mmol) and (2-(2-chloro)ethoxy)ethanol (1.46 mmol) were added to the reaction mixture and reaction mixture was heated to 78° C. for 5 h. The reaction mixture was cooled, diluted with water (100 mL), and extracted with ethyl acetate (100 mL). The organic layer was washed with water (3×60 mL) and then with brine. The organic layer was then dried over anhydrous Na₂SO₄ and filtered. The filtrate was concentrated and purified using flash column chromatography (using 2-5% MeOH: DCM) to obtain product (69% yield). 1H NMR (DMSO-d₆, 600 MHZ): δ 11.13 (s, 1H), 8.70 (d, J=8.34 Hz, 1H), 7.65 (d, J=7.62 Hz, 1H), 7.58 (d, J=7.68 Hz, 1H), 7.40-7.42 (m, 2H), 7.26 (d, J=8.22 Hz, 1H), 7.03 (t, J=7.50 Hz, 1H), 4.55-4.57 (m, 1H), 3.99 (t, J=5.40 Hz, 2H), 3.67 (t, J=5.64 Hz, 2H), 3.44-3.45 (m, 4H); ¹³C NMR (DMSO-d₆, 100 MHZ): δ 188.6, 168.9, 152.4, 142.6, 139.0, 137.2, 125.5, 124.5, 124.2, 121.6, 121.5, 119.8, 119.0, 113.6, 112.0, 104.1, 72.0, 67.5, 60.1; HRMS for [C₂₀H₁₇⁷⁹BrN₂O₄+H]⁺: Calcd. 429.0450, Found: 429.0417

### Synthesis of (2Z,3E)-6′-bromo-1-(2-(2-hydroxyethoxy)ethyl)-3-(hydroxyimino)-[2,3′-biindolinylidene]-2′-one (f)

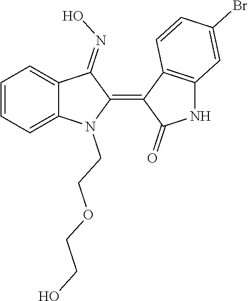 [SMILES: O=C1Nc2cc(Br)ccc2/C1=C1\C(=N\O)c2ccccc2N1CCOCCO]

In a 25 mL 2-neck round bottom flask 1 (0.139 mmol) and hydroxylammonium hydrochloride (1.39 mmol) were added under argon. To this mixture dry pyridine (1 mL) was added and reaction mixture was refluxed at 120° C. for 1.5 h. The reaction mixture was cooled, diluted with water (100 mL), and extracted with ethyl acetate (3×50 mL). The organic layer was washed with water (3×30 mL) and then with brine. The organic layer was then dried over anhydrous Na₂SO₄ and filtered. The filtrate was concentrated and purified using flash column chromatography (using 2-4% MeOH: DCM) to obtain product (96% yield) 1H NMR (DMSO-d₆, 400 MHZ): δ 13.65 (s, 1H), 11.78 (s, 1H), 8.60 (d, J=8.4 Hz, 1H), 8.22 (d, J=7.6 Hz, 1H), 7.41-7.44 (m, 2H), 7.38 (d, J=1.80 Hz, 1H), 7.13-7.15 (dd, J=8.4, 1.80 Hz, 1H), 7.03-7.07 (m, 1H), 4.52-4.55 (m, 1H), 4.03 (t, J=5.68 Hz, 2H), 3.68 (t, J=5.6 Hz, 2H), 3.44-3.45 (m, 4H); ¹³C NMR (DMSO-d₆, 100 MHz): δ 168.8, 151.2, 146.1, 144.6, 140.3, 132.1, 127.9, 123.8, 123.0, 121.9, 121.1, 118.0, 116.4, 111.8, 111.1, 96.7, 72.1, 67.7, 60.1; HRMS for [C₂₀H₁₈⁷⁹BrN₃O₄+H]⁺: Calcd. 444.0559, Found: 444.0518. While working up the reaction for pegylated 6-bromoindirubin derivatives extraction was done instead of filtration. Since the product was more soluble in aqueous layer, during filtration the product would go in the aqueous layer too.

Similarly, (2Z,3E)-1-(2-hydroxyethyl)-3-(hydroxyimino)-6′-bromo-[2,3′-biindolinylidene]-2′-one lithium salt (compound n) was prepared.

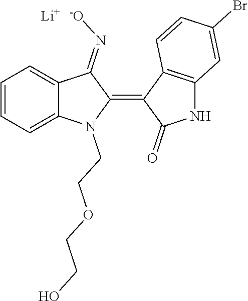

### Synthesis of (Z)-6′-bromo-1-(2-(2-(2-hydroxyethoxy)ethoxy)ethyl)-[2,3′-biindolinylidene]-2′,3-dione (2)

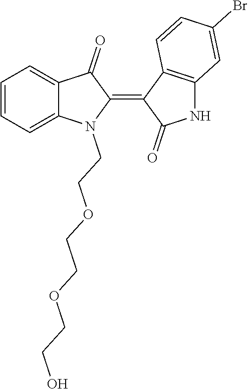 [SMILES: O=C1Nc2cc(Br)ccc2/C1=C1\C(=O)c2ccccc2N1CCOCCOCCO]

In a 25 mL 2-neck round bottom flask 6-bromoindirubin (0.48 mmol) and potassium carbonate (1.44 mmol) were dissolved in dry DMF (10 mL) were added under argon. To this solution 2-[2-(2-chloroethoxy)ethoxy]ethanol (4.8 mmol) was added drop wise and reaction mixture was stirred at 65° C. overnight. The reaction mixture was cooled, diluted with water (100 mL), and then filtered. The residue was transferred to a round bottom flask and then dried on high vacuum to obtain pure product (63% yield). ¹H NMR (DMSO-d₆, 400 MHz): δ 11.13 (s, 1H), 8.70 (d, J=8.36 Hz, 1H), 7.65 (d, J=7.48 Hz, 1H), 7.57-7.61 (m, 1H), 7.40-7.42 (m, 2H), 7.25-7.27 (dd, J=8.36, 1.76 Hz, 1H), 7.04 (t, J=7.44 Hz, 1H), 4.50 (t, J=5.48 Hz, 1H), 4.00 (t, J=5.44 Hz, 2H), 3.67 (t, J=5.36 Hz, 2H), 3.52-3.54 (m, 2H), 3.43-3.46 (m, 2H), 3.40 (t, J=5.36 Hz, 2H), 3.34 (t, J=5.12 Hz; HRMS for [C₂₂H₂₁⁷⁹BrN₂O₅+H]⁺: Calcd. 473.0712, Found: 473.0708.

### Synthesis (2Z,3E)-6′-bromo-1-(2-(2-(2-hydroxyethoxy)ethoxy)ethyl)-3-(hydroxyimino)-[2,3′-biindolinylidene]-2′-one (g)

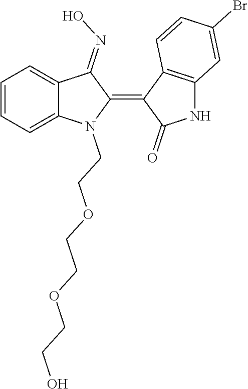 [SMILES: O=C1Nc2cc(Br)ccc2/C1=C1\C(=N\O)c2ccccc2N1CCOCCOCCO]

In a 25 mL 2-neck round bottom flask 2 (0.295 mmol) and hydroxylammonium hydrochloride (2.95 mmol) were added under argon. To this mixture dry pyridine (5 mL) was added and reaction mixture was refluxed at 120° C. for 1.5 h. The reaction mixture was cooled, diluted with water (100 mL), and then filtered. The organic layer was then dried over anhydrous Na₂SO₄ and filtered. The crude product was purified using flash column chromatography (using 0.5-5% MeOH: DCM) to obtain product (67% yield). ¹H NMR (DMSO-d₆, 400 MHZ): δ 13.66 (s, 1H), 11.78 (s, 1H), 8.60 (d, J=8.44 Hz, 1H), 8.22 (d, J=7.72 Hz, 1H), 7.41-7.44 (m, 2H), 7.38 (s, 1H), 7.13-7.15 (d, J=8.24, 1H), 7.04-7.07 (m, 1H), 4.51 (t, J=5.44 Hz, 1H), 4.04 (t, J=5.40 Hz, 2H), 3.68 (t, J=5.44 Hz, 2H), 3.52-3.55 (m, 2H), 3.44-3.47 (m, 2H), 3.41 (t, J=5.20 Hz, 2H), 3.35 (t, J=5.12 Hz, 2H); ¹³C NMR (DMSO-d₆, 100 MHz): δ 168.9, 151.3, 146.2, 144.7, 140.2, 132.2, 127.9, 123.9, 123.1, 122.0, 121.1, 118.0, 116.4, 111.8, 111.3, 96.8, 72.4, 69.8, 69.7, 67.9, 60.2; HRMS for [C₂₂H₂₂⁷⁹BrN₃O₅+H]⁺: Calcd.: 488.0821, Found: 488.0787. A white solid was obtained after triturating the oily compound obtained after concentrating the column fractions containing the desired product.

### Synthesis of benzyl 3-(((E)-((Z)-6′-bromo-2′-oxo-[2,3′-biindolinylidene]-3-ylidene)amino)oxy)-2-((((Z)—((Z)-6′-bromo-2′-oxo-[2,3′-biindolinylidene]-3-ylidene)amino)oxy)methyl)-2-methylpropanoate (h)

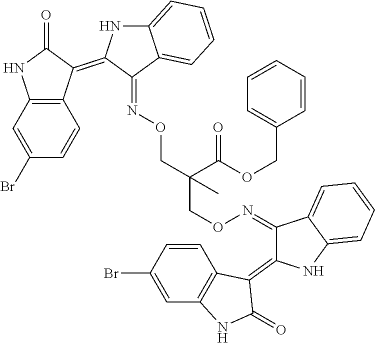 [SMILES: CC(CO/N=C1\C(=C2\C(=O)Nc3cc(Br)ccc32)Nc2ccccc21)(CO/N=C1/C(=C2/C(=O)Nc3cc(Br)ccc32)Nc2ccccc21)C(=O)OCc1ccccc1]

In a 25 mL 2-neck round bottom flask under argon, lithium salt of 6-bromoindirubin oxime (0.138 mmol) was dissolved in dry DMF (3 mL). To this solution, compound benzyl 2-methyl-3-(tosyloxy)-2-((tosyloxy) methyl) propanoate (0.069 mmol) and potassium carbonate (0.138 mmol) were added and reaction mixture was allowed to stir at room temperature for 20 h. Potassium carbonate (0.138 mmol) was again added to the reaction mixture and stirred at room temperature for next 24 h. The reaction mixture was diluted with water (50 mL) and extracted using ethyl acetate. It was possible to filter the diluted reaction mixture hence extraction was done. The organic layer was washed with brine, dried over sodium sulphate, and then filtered. The filtrate was concentrated and purified using flash column chromatography. During the column the monomer and dimer of 6-Bio were isolated.

The monomer of 6-bromoindirubin oxime was again reacted with 6-bromoindirubin oxime to obtain the dimer in good yields. In a 25 mL 2-neck round bottom flask under argon, monomer (0.053 mmol), 6-bromoindirubin oxime (0.053 mmol) and potassium carbonate (0.106 mmol) were dissolved in dry DMF (3 mL) and the reaction mixture was heated to 50° C. for 20 h. The temperature was then raised to 70° C. and stirred for 1.5 h. To this potassium carbonate (0.724 mmol) was added and reaction was stirred at 70° C. for 3 h more. The reaction mixture was cooled and diluted with water (100 mL) and extracted with ethyl acetate (3×15 mL). The combined organic layer was given brine wash, dried over sodium sulphate, and then filtered to obtain crude product. Dimer was purified using flash column chromatography (using 1-5% MeOH: DCM as eluant) with 29% yield. 1H NMR (DMSO-d₆, 400 MHZ): δ 11.43 (s, 2H), 10.72 (s, 2H), 8.24 (d, J=8.40 Hz, 2H), 7.95 (d, J=7.60 Hz, 2H), 7.39 (t, J=7.96 Hz, 2H), 7.18-7.32 (m, 8H), 6.89-6.92 (m, 4H), 6.56-6.59 (dd, J=8.44, 1.84 Hz, 2H), 5.21 (s, 2H), 4.96 (s, 4H), 1.57 (s, 3H); ¹³C NMR (DMSO-d₆, 100 MHz): δ 171.4, 169.8, 145.2, 135.5, 131.4, 130.2, 128.4, 128.1, 127.8, 127.5, 69.8, 66.2, 65.7, 46.2, 45.9, 21.0, 16.7, 16.2; HRMS for [C₄₄H₃₂⁷⁹Br₂N₆O₆+H]⁺: Calcd. 901.0808, Found: 901.0740.

### Synthesis of 2-((benzyloxy)carbonyl)-2-methylpropane-1,3-diyl bis(2-methyl-3-(tosyloxy)-2-((tosyloxy)methyl)propanoate) (3)

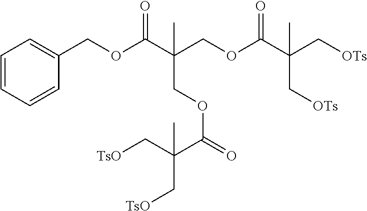 [SMILES: Cc1ccc(S(=O)(=O)OCC(C)(COS(=O)(=O)c2ccc(C)cc2)C(=O)OCC(C)(COC(=O)C(C)(COS(=O)(=O)c2ccc(C)cc2)COS(=O)(=O)c2ccc(C)cc2)C(=O)OCc2ccccc2)cc1]

In a 50 mL 2-neck round bottom flask under argon, compound 2-((benzyloxy)carbonyl)-2-methylpropane-1,3-diyl bis(3-hydroxy-2-(hydroxymethyl)-2-methylpropanoate) (0.438 mmol), pyridine (2.62 mmol) and 4-dimethylaminopyridine (2.62 mmol) were dissolved in dry DCM (10 mL). To the reaction mixture, a solution of freshly recrystallized p-toluenesulfonyl chloride (2.62 mmol) in dry DCM (4 mL) was added dropwise over a period of 5 min. The reaction was allowed to stir at room temperature for 17 h. To the reaction p-toluenesulfonyl chloride (2.62 mmol) was added again and reaction was stirred at room temperature for another overnight. Water (100 mL) was added to the reaction mixture and extraction was done using DCM (3×25 mL). The combined organic layer was given brine wash, dried over sodium sulphate, and then filtered. The filtrate was concentrated to obtain crude product which was purified using flash column chromatography (in 30-50% ethyl acetate—pet ether as eluant) with 74% yield [after n-pentane wash (3×10 mL ¹H NMR (DMSO-d₆, 400 MHZ): δ 7.70 (d, J=8.28 Hz, 8H), 7.45 (d, J=8.20 Hz, 8H), 7.27-7.34 (m, 5H), 5.03 (s, 2H), 3.94-4.08 (m, 12H), 2.40 (s, 12H), 1.02 (s, 3H). 0.93 (s, 6H); ¹³C NMR (DMSO-d₆, 100 MHz): δ 171.4, 169.8, 145.2, 135.5, 131.4, 130.2, 128.4, 128.1, 127.8, 127.5, 69.8, 66.2, 65.7, 46.2, 45.9, 21.0, 16.7, 16.2; Mass was not taken for the compound.

### Synthesis of 2-((benzyloxy)carbonyl)-2-methylpropane-1,3-diyl bis(3-((((2Z,3E)-6′-bromo-2′-oxo-[2,3′-biindolinylidene]-3-ylidene)amino)oxy)-2-(((((2Z,3E)-6′-bromo-2′-oxo-[2,3′-biindolinylidene]-3-ylidene)amino)oxy)methyl)-2-methylpropanoate (i)

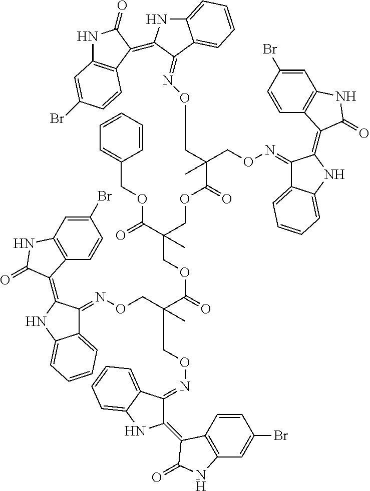 [SMILES: CC(CO/N=C1/C(=C2/C(=O)Nc3cc(Br)ccc32)Nc2ccccc21)(CO/N=C1/C(=C2/C(=O)Nc3cc(Br)ccc32)Nc2ccccc21)C(=O)OCC(C)(COC(=O)C(C)(CO/N=C1/C(=C2/C(=O)Nc3cc(Br)ccc32)Nc2ccccc21)CO/N=C1/C(=C2/C(=O)Nc3cc(Br)ccc32)Nc2ccccc21)C(=O)OCc1ccccc1]

In a 25 mL 2-neck round bottom flask under argon, compound 3 (0.251 mmol), 6-bromoindirubin oxime (1.257 mmol) and potassium carbonate (1.509 mmol) were dissolved in dry DMF (10 mL). The reaction mixture was heated to 70° C. for 48 h. The reaction mixture was cooled, diluted with water (100 mL), and then filtered. The residue was washed with water (50 mL) and then transferred to round bottom flask to obtain crude product. The crude product was then washed with ethyl acetate (5×15 mL), sonicated, and then decanted. The residue was dried on high vacuum with heating (80° C.) for 30 h then characterised by NMR to be tetramer of 6-BIO, i. ¹H NMR (DMSO-d₆, 400 MHZ): δ 11.35 (s, 4H), 10.65 (s, 4H), 8.13 (d, J=8.44 Hz, 4H), 7.91 (d, J=7.76 Hz, 4H), 7.33 (t, J=7.76 Hz, 4H), 7.16-7.26 (m, 9H), 6.91 (t, J=7.64 Hz, 4H), 6.82 (t, J=2.08 Hz, 4H), 6.47-6.51 (dt, J=8.28, 2.32 Hz, 4H), 4.98 (s, 2H), 4.73-4.84 (m, 8H), 4.38 (s, 4H), 1.39 (s, 6H), 1.20-1.23 (m, 9H); ¹³C NMR (DMSO-d₆, 100 MHZ): δ 172.7, 171.9, 170.4, 151.4, 145.3, 143.8, 139.6, 135.4, 132.7, 128.3, 128.0, 127.8, 127.6, 124.3, 122.3, 121.3, 121.1, 118.2, 115.9, 111.8, 111.5, 111.2, 99.3, 78.2, 78.1, 48.1, 46.2. 17.6; MALDI for [C₈₆H₆₄⁷⁹Br₄N₁₂O₁₄]⁺: Calcd. 1809.1164, Found: 1809.177. Attempts were done to purify the crude product using flash column chromatography in MeOH: DCM solvent system. But mixture was obtained all the time. Hence washing with ethyl acetate was done to remove 6-bromoindirubin and other impurities.

### Synthesis of (2Z,3E)-1-(2-(2-(2-hydroxyethoxy)ethoxy)ethyl)-3-(hydroxyimino)-6′-((E)-styryl)-[2,3′-biindolinylidene]-2′-one (j)

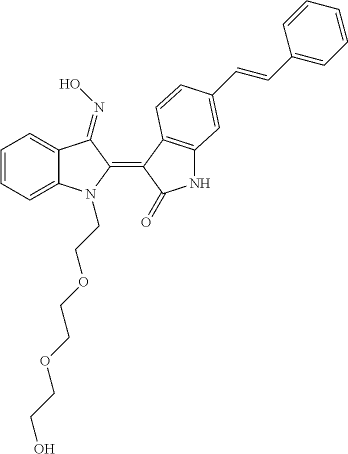 [SMILES: O=C1Nc2cc(/C=C/c3ccccc3)ccc2/C1=C1\C(=N\O)c2ccccc2N1CCOCCOCCO]

In a 25 mL 2-neck round bottom flask, compound g (0.163 mmol), palladium acetate (0.049 mmol) and tri(o-tolyl)phosphine (0.121 mmol) were added under argon and subjected to three cycles of vacuum pump-argon cycle. To the mixture, styrene (0.344 mmol) and triethylamine (10 mL) were sequentially added and then refluxed at 110° C. for 5 h. The reaction mixture was cooled and triethylamine was evaporated to obtain residue which was purified by flash column chromatography (using 1-2% MeOH: DCM as eluant). NMR data indicated some impurity accompanying the product. Subsequent flash column chromatography to get rid of the impurity was also in vain. The compound needs to be purified by preparatory HPLC. Crude). ¹H NMR (DMSO-d₆, 400 MHz): δ 13.56 (s, 1H), 11.75 (s, 1H), 8.68 (d, J=8.12 Hz, 1H), 8.24 (d, J=7.76 Hz, 1H), 7.62 (d, J=7.36, 2H), 7.20-46 (m, 9H), 7.02-7.06 (m, 1H), 4.51 (t, J=5.32, 1H), 4.10 (t, J=5.84, 2H), 3.74 (t, J=5.76, 2H), 3.34-3.59 (m, 8H); HRMS for [C₃₀H₂₉N₃O₅+H]⁺: Calcd.: 512.2185, Found: 512.2153.

### Synthesis of (2Z,3E)-6′-bromo-3-((2-(2-(2-hydroxyethoxy)ethoxy)ethoxy)imino)-1-(2-(2-(2-hydroxyethoxy)ethoxy)ethyl)-[2,3′-biindolinylidene]-2′-one (k)

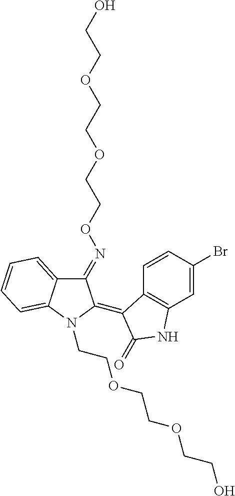 [SMILES: O=C1Nc2cc(Br)ccc2/C1=C1\C(=N\OCCOCCOCCO)c2ccccc2N1CCOCCOCCO]

In a 50 mL 2-neck round bottom flask, 6-bromoindirubin oxime (0.505 mmol) and potassium carbonate (1.516 mmol) were dissolved in dry DMF (10 mL) under argon. To this mixture 2-[2-(2-chloroethoxy)ethoxy]ethanol (5.053 mmol) was added and reaction mixture was stirred at 75° C. for 18 h. The reaction mixture was cooled, diluted with water (200 mL), and then extracted with ethyl acetate (3×50 mL). The combined organic layer was washed brine, dried over sodium sulphate, and filtered. The filtrate was concentrated and then purified using flash column chromatography (using 1-4% MeOH: DCM as eluant) to obtain pure product (quantitative yield). The reaction mixture was cooled, diluted with water (200 mL), and then filtered. The organic layer was then dried over anhydrous Na₂SO₄ and filtered. The crude product was purified using flash column chromatography (using 0.5-5% MeOH: DCM) to obtain product (36% yield). ¹H NMR (DMSO-d₆, 400 MHz): δ 11.69 (s, 1H), 8.53 (d, J=8.40 Hz, 1H), 8.12 (d, J=7.56 Hz, 1H), 7.42-7.45 (m, 2H), 7.35 (s, 1H), 7.18 (d, J=8.24, 1H), 7.04 (t, J=6.40 Hz, 1H), 4.51 (bs, 2H), 4.50-4.55 (m, 2H), 4.01 (bs, 2H), 3.91 (bs, 2H), 3.61-3.67 (m, 4H), 3.53 (bs, 4H), 3.35-3.45 (m, 10H); ¹³C NMR (DMSO-d₆, 100 MHz): δ 168.7, 151.2, 145.2, 144.6, 140.6, 132.9, 128.3, 124.2, 123.4, 121.8, 120.6, 118.4, 116.1, 111.9, 111.2, 98.0, 76.0, 72.4, 72.3, 70.0, 69.8, 69.7, 69.7, 68.7, 67.8, 60.1; HRMS for [C₂₈H₃₄⁷⁹BrN₃O₈+H]⁺: Calcd.: 620.1608, Found: 620.1573.

### Synthesis of: (2Z,3E)-3-(hydroxyimino)-6′-nitro-[2,3′-biindolinylidene]-2′-one (1)

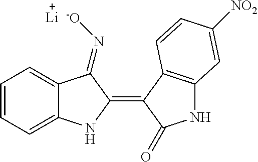

To a solution of 6-nitroindirubin oxime (0.364 mmol) in THF (10 mL, HPLC grade), 2.5 mL of aqueous solution of lithium hydroxide (0.364 mmol) was added dropwise and the reaction mixture was stirred at room temperature for 10 min. The solvent was evaporated, washed with n-pentane (3×10 mL), and then dried on hi-vacuum to obtain quantitative yield of product. ¹H NMR (DMSO-d₆, 400 MHz): δ 12.69 (s, 1H), 10.68 (s, 1H), 8.65 (d, J=8.88 Hz, 1H), 8.32 (d, J=7.64 Hz, 1H), 7.74-7.77 (dd, J=8.80, 2.28 Hz, 1H), 7.62 (d, J=2.24 Hz, 1H), 7.40 (d, J=7.72 Hz, 1H), 7.14 (t, J=6.76 Hz, 1H), 7.03 (t, J=6.52 Hz, 1H).

### Synthesis of (2Z,3E)-6′-bromo 3-((2-hydroxyethoxy)imino)-[2,3′-biindolinylidene]-2′-one (m)

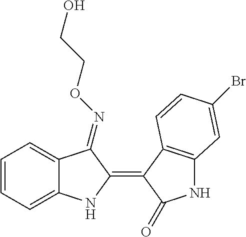 [SMILES: O=C1Nc2cc(Br)ccc2/C1=C1/Nc2ccccc2/C1=N\OCCO]

In a 25 mL 2-neck round bottom flask 6-bromoindirubin oxime (0.14 mmol) and potassium carbonate (0.168 mmol) were added under argon. To this mixture dry DMF (3 mL) was added and reaction mixture was stirred for 5 min. 2-bromoethanol (1.40 mmol) was then added and reaction mixture was stirred at room temperature for 18 h. The reaction mixture was diluted with water (50 mL) and filtered. The residue was washed with more water and then dried. The residue was washed with diethyl ether (3-4 times, 10 mL) and decanted. The precipitate was then dried to obtain pure product (58% yield). ¹H NMR (DMSO-d₆, 600 MHZ): δ 11.70 (s, 1H), 10.89 (s, 1H), 8.54 (d, J=8.58 Hz, 1H), 8.18 (d, J=7.74 Hz, 1H), 7.43-7.45 (m, 2H), 7.15 (d, J=8.10 Hz, 1H), 7.04-7.06 (m, 1H), 7.02 (s, 1H), 5.00 (t, J=5.34 Hz, 1H), 4.61 (t, J=4.62 Hz, 2H), 3.89 (q, J=5.1 Hz, 2H); 13C NMR (DMSO-d₆, 100 MHZ): δ 170.6, 151.2, 145.3, 144.6, 139.9, 132.8, 128.4, 124.5, 123.1, 121.7, 121.6, 118.2, 116.2, 111.9, 111.5, 98.8, 78.5, 59.4; HMRS for [C₁₈H₁₄⁷⁹BrN₃O₃+H]⁺: Calcd. 400.0297, Found: 400.0256

### Synthesis of (2Z,3E)-3-(hydroxyimino)-6′-((E)-styryl)-[2,3′-biindolinylidene]-2′-one (o)

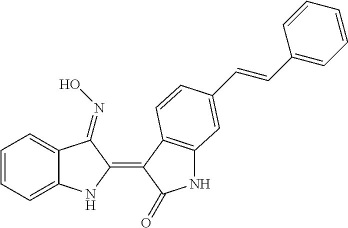 [SMILES: O=C1Nc2cc(/C=C/c3ccccc3)ccc2/C1=C1/Nc2ccccc2/C1=N\O]

A 25 mL round bottom flask is charged with 50 mg of 6-bromo indirubin oxime (0.14), 9.5 mg of Pd(OAc)₂ and 31.6 mg of tri (o-tolyl)phosphine. The flask was evacuated and back-filled with argon thrice. Following this, 34 μL of styrene and 10 mL of triethylamine. The round bottom flask was then immersed in an oil bath maintained at 105° C. for 18 h. The reaction is cooled to room temperature and triethyl amine is removed on a rotary evaporator. The residue is purified by column chromatography to yield 26 mg of product.

### Synthesis of 6-nitroindirubin

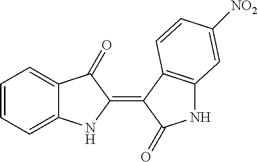 [SMILES: O=C1Nc2cc([N+](=O)[O-])ccc2/C1=C1/Nc2ccccc2C1=O]

In a 10 mL, 2-neck round bottom flask under dry argon, dry methanol (20 mL) was added. Methanol was vigorously stirred, and argon was bubbled through it for 20 min. 6-nitro isatin (0.468 mmol) and 3-indoxyl acetate (0.468 mmol) were then added sequentially to methanol and allowed to stir for 5 min. To this solution anhydrous sodium bicarbonate (1.17 mmol) was added. The reaction was allowed to stir at room temperature for 4.5 h. The reaction mixture was then filtered and washed with water (50 mL) and then with 1:1 methanol-water solution (100 mL). The residue was purified using flash column chromatography (using 1% MeOH:DCM as eluant) to obtain product in 38% yield. ¹H NMR (DMSO-d₆, 600 MHz): δ 11.44 (s, 1H), 11.28 (s, 1H), 8.89 (d, J=8.88 Hz, 1H), 7.95-7.96 (m, 1H), 7.69 (d, J=7.50 Hz, 1H), 7.60-7.63 (m, 2H); 7.47 (d, J=7.74 Hz, 1H), 7.09 (t, J=7.32 Hz, 1H).

(2Z,3E)-3-(hydroxyimino)-6′-nitro-[2,3′-biindolinylidene]-2′-one (p) was synthesized by reacting 6-nitroindirubin with hydroxylammonium hydrochloride.

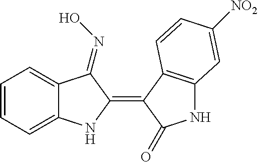 [SMILES: O=C1Nc2cc([N+](=O)[O-])ccc2/C1=C1/Nc2ccccc2/C1=N\O]

### Synthesis of 6-Methoxy Isatin

In a 250 mL round bottom flask, m-anisidine (15.83 mmol), aqueous HCl (I mL in 9 mL of water), hydroxylamine hydrochloride (51.08 mmol) and sodium sulphate (94.97 mmol) were dissolved in water (50 mL) and heated to boiling. To this boiling solution a solution of chloral hydrate (18.92 mmol) was added and the solution was boiled for 1 h and then allowed to cool gradually to room temperature. The reaction mixture was extracted with ethyl acetate (3×150 mL). The organic layer was given brine wash, dried over sodium sulphate, and filtered. The filtrate was evaporated to obtain crude product which was purified by flash column chromatography (using 20-40% ethyl acetate-pet ether as eluant). Product with slight non-polar impurities was washed with diethyl ether to obtain 0.256 g of pure isonitrosoacetanilide intermediate. The isonitrosoacetanilide intermediate (purified by flash column chromatography) was added in portions to a solution of concentrated sulphuric acid (2 mL) and water (0.2 mL) and warmed to 65° C. for 15 min and then to 74° C. for 50 min. Reaction mixture was diluted with water (100 mL) and the aqueous phase was extracted with ethyl acetate (3×25 mL). The organic layer was given brine wash, dried over sodium sulphate, and filtered. The filtrate was evaporated to obtain crude product which was purified by flash column chromatography (using 20-40% ethyl acetate-pet ether as eluant) to obtain 6-methoxy isatin (8.7% yield).

### Synthesis of 6-Methoxy indirubin

In a 25 mL, 2-neck round bottom flask under dry argon, dry methanol (6 mL) was added. Methanol was vigorously stirred and argon was bubbled through it for 20 min. 6-Methoxy isatin (0.129 mmol) and 3-indoxyl acetate (0.129 mmol) were then added sequentially to methanol and allowed to stir for 5 min. To this solution anhydrous sodium bicarbonate (0.0.324 mmol) was added. The reaction was allowed to stir at room temperature for 3 h. The reaction mixture was then filtered and washed with 1:1 methanol-water solution (25 mL) and then with water (25 mL) to obtain crude product. Pure product was obtained by using flash column chromatography (in 0.5% MeOH: DCM as eluant) with 70% yield. ¹H NMR (DMSO-d₆, 400 MHZ): δ 10.85 (s, 1H), 10.78 (s, 1H), 8.73 (d, J=8.72 Hz, 1H), 7.63 (d, J=7.44 Hz, 1H), 7.54 (t, J=7.36 Hz, 1H), 7.38 (d, J=8.16 Hz, 1H), 6.98 (t, J=7.40 Hz, 1H), 6.58-6.61 (dd, J=8.76, 2.16 Hz, 1H), 6.47 (d, J=2.08 Hz, 1H), 3.79 (s, 3H).

### Synthesis of (2Z,3E)-3-(hydroxyimino)-6′-methoxy-[2,3′-biindolinylidene]-2′-one (6-MIO)

In a 25 mL 2-neck round bottom flask, 6-methoxy indirubin (0.0821 mmol) and hydroxylammonium hydrochloride (0.821 mmol) were added under argon. To this mixture dry pyridine (3 mL) was added and reaction mixture was refluxed at 120° C. for 1.5 h. The reaction mixture was cooled, diluted with water (100 mL), and then extracted with ethyl acetate (3×25 mL). The combined organic layer was washed brine, dried over sodium sulphate, and filtered. The filtrate was concentrated and then purified using flash column chromatography (using 0.5-1.5% MeOH: DCM as eluant) to obtain pure product (quantitative yield). ¹H NMR (DMSO-d₆, 400 MHZ): δ 13.28 (s, 1H), 11.45 (s, 1H), 10.67 (s, 1H), 8.56 (d, J=8.44 Hz, 1H), 8.22 (d, J=7.64 Hz, 1H), 7.35-7.40 (m, 2H), 6.96-7.00 (m, 1H), 6.48-6.51 (m, 2H), 3.77 (s, 3H); ¹³C NMR (CDCl₃, 100 MHz): δ 171.4, 158.4, 151.2, 145.0, 143.0, 140.0, 131.9, 127.9, 124.2, 120.9, 116.5, 115.7, 111.2, 105.9, 99.2, 95.3, 55.1.

### Autophagic Flux

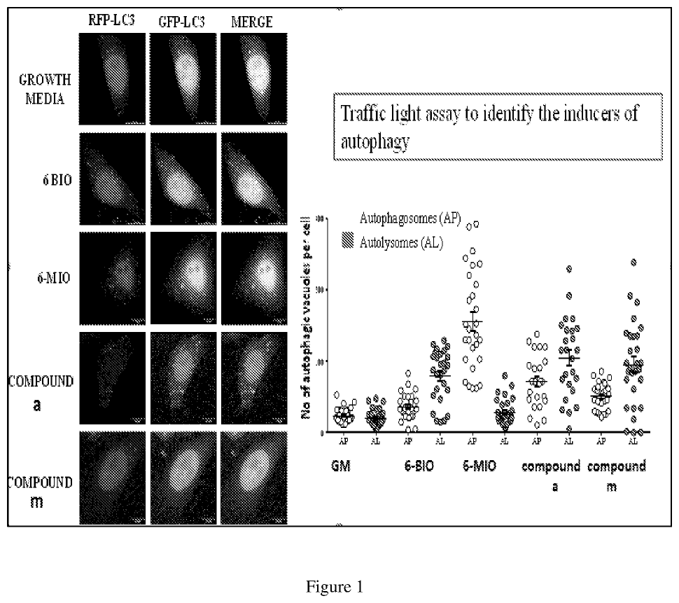

Autophagic flux refers to a measure of autophagic degradation activity. FIG. 1 depicts the fluorescent analysis of the autophagic flux in the presence of 6-bromoindirubin oxime (6-BIO), 6-MIO, and compounds of Formula I—compounds ‘a’ and ‘m’. FIG. 1 depicts that both autophagosomes and autolysosomes are increased with the compounds of Formula I when compared to the growth media control.

The assays were essentially performed as follows: RFP-EGFP-LC3 assay: sub-confluent HeLa and/or SH-SY5Y cells were seeded into 60 mm cell culture dishes, then transfected with ptf LC3 construct and/or siRNA and allowed to express for 48 h. Cells were trypsinized, seeded again on poly-D-lysine coated cover slips in a 12 or 24 well plates and allowed to attach. After appropriate treatments, the coverslips containing cells were processed for imaging. For immunofluorescent antibody staining, the cover slips were incubated in primary antibody at 4° C. for overnight followed by secondary antibody incubation at room temperature. For imaging the mammalian cells, after appropriate treatments, coverslips containing cells were fixed using 4% paraformaldehyde (PFA; Sigma) and then permeabilized using Triton X-100 (0.2%, HiMedia). Coverslips were mounted on slide using antifade, Vectashield mounting medium (Vector Laboratories). Images were acquired using DeltaVision Elite widefieldmicroscope (API, GE) with following filters: FITC (490/20 and 529/38), TRITC (542/27 and 594/45) and Cy5 (632/22 and 676/34). Acquired images were processed using DV softWoRX software. In this standard assay, the autophagosomes would appear as yellow whereas autolysosomes would be red as EGFP gets quenched at lysosomal acidic pH6.

It can be clearly seen that the compounds of Formula I induces autophagy and strongly drives autophagy flux resulting in aggregate clearance in comparison to 6-bromoindirubin oxime (6-BIO). The compounds of Formula I modulates autophagy flux through inhibiting GSK3β activity. Compounds of the Formula I disclosed here show enhanced solubility compared to 6-BIO and have similar or better efficacy in modulating autophagy flux

The compounds of Formula I crosses the blood brain barrier and correct the synaptic deficits and helps in restoration of the synaptic function. Thus, the compounds of Formula I are potential candidate in neurodevelopmental therapeutics and provides the possibility that cognitive, emotional, and social symptoms that result from hard-wired neuronal circuit damage during development may still be corrected even by late pharmacological intervention.

### ADVANTAGES OF THE PRESENT DISCLOSURE

The present disclosure provides indirubin compounds of Formula I and its polymorphs, stereoisomers, prodrugs, solvates, co-crystals, intermediates, pharmaceutically acceptable salts, and metabolites thereof. The present disclosure also provides a process of preparation of the compounds of Formula I. The compounds of Formula I are small molecules that act as GSK3β inhibitors. The compounds of Formula I induces autophagy and strongly drives autophagy flux resulting in aggregate clearance. The compounds of Formula I modulates autophagy flux through inhibiting GSK3β activity and results in increased autophagy. The compounds of Formula I helps in restoration of the synaptic function. The present disclosure further provides a pharmaceutical composition and a method of treating or preventing a disease or disorder. The compounds of Formula I are capable of crossing the blood brain barrier and can rectify the synaptic deficits and thus prevents/treats intellectual disability (ID) or autism spectrum disorder, functional, behavioral, or neurodegenerative disorders. The compounds of Formula I are also useful in treating/preventing diseases such as Alzhemier's, Parkinson's, Amyotrophic Lateral Sclerosis Ataxia, Stroke, Migraine, Muscular Dystrophy, Multiple Sclerosis, Bell's Palsy, Epilepsy and Seizures, Guillain-Barré Syndrome, bipolar disorders, and other diseases such as diabetes mellitus, cancer and so on. The compounds of Formula I are potential small molecule drug candidate useful in neurodevelopmental therapeutics.

1. A compound of Formula I, 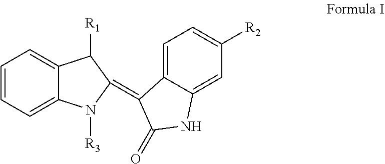
and its polymorphs, stereoisomers, prodrugs, solvates, co-crystals, intermediates, pharmaceutically acceptable salts, and metabolites thereof,
wherein R₁ is ═N—O-A, wherein A is selected from hydrogen, C₁₋₁₀ alkyl, C₂₋₁₀ alkenyl, C₂₋₁₀ alkynyl, C₆₋₁₄ aryl, or —((CH₂)ₘ—O—)ₙH, and wherein C₁₋₁₀ alkyl, C₂₋₁₀ alkenyl, C₂₋₁₀ alkynyl, or C₆₋₁₄ aryl is optionally substituted with one or more groups selected from C₁₋₁₀ alkyl, C₂₋₁₀ alkenyl, C₂₋₁₀ alkynyl, C₆₋₁₄ aryl, —B, or —COO—C₁₋₁₀ alkyl-D, wherein —COO—C₁₋₁₀ alkyl-D is optionally substituted with —OCO—C₁₋₁₀ alkyl-E, or —COO—C₁₋₁₀ alkyl-E, wherein —OCO—C₁₋₁₀ alkyl-E, or —COO—C₁₋₁₀ alkyl-E is optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₁₀ alkyl, C₂₋₁₀ alkenyl, C₂₋₁₀ alkynyl, C₆₋₁₄ aryl, —COO—CH₂—C₆₋₁₄ aryl, C₁₋₂₀ heterocyclyl, or —O—N—C₁₋₂₀ heterocyclyl and wherein C₁₋₂₀ heterocyclyl, —O—N—C₁₋₂₀ heterocyclyl has one or more heteroatoms selected from nitrogen, oxygen, or sulphur, and is optionally further substituted;
R₂ is selected from hydrogen, halogen, C₁₋₁₀ alkyl, C₂₋₁₀ alkenyl, C₂₋₁₀ alkynyl, C₆₋₁₄ aryl, nitro, amine, or amide, wherein C₁₋₁₀ alkyl, C₂₋₁₀ alkenyl, or C₆₋₁₄ aryl is optionally substituted with one or more C₆₋₁₄ aryl, or halogen;
R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH;
m is 1 to 3; and n is 1 to 10.

2. The compound as claimed in claim 1, wherein R₁ is ═N—O-A, wherein A is selected from hydrogen, C₁₋₈ alkyl, C₂₋₆ alkenyl, C₂₋₆ alkynyl, C₆₋₁₀ aryl, or —((CH₂)ₘ—O—)ₙH, and wherein C₁₋₈ alkyl, C₂₋₆ alkenyl, C₂₋₆ alkynyl, or C₆₋₁₀ aryl is optionally substituted with one or more groups selected from C₁₋₈ alkyl, C₂₋₆ alkenyl, C₂₋₆ alkynyl, C₆₋₁₀ aryl, —B, or —COO—C₁₋₈ alkyl-D, wherein —COO—C₁₋₈ alkyl-D is optionally substituted with —OCO—C₁₋₁₀ alkyl-E optionally substituted with —F, wherein B is selected from —COO—CH₂—C₆₋₁₄ aryl, C₁₋₁₈ heterocyclyl, or —O—N—C₁₋₁₈ heterocyclyl, wherein C₁₋₁₈ heterocyclyl, —O—N—C₁₋₁₈ heterocyclyl has one or more heteroatoms selected from nitrogen, oxygen, or sulphur, and is optionally further substituted; wherein D, E, and F are independently selected from C₁₋₈ alkyl, C₂₋₆ alkenyl, C₆₋₁₄ aryl, —COO—CH₂—C₆₋₁₄ aryl, C₁₋₁₈ heterocyclyl, or —O—N—C₁₋₁₈ heterocyclyl, wherein C₁₋₁₈ heterocyclyl, —O—N—C₁₋₁₈ heterocyclyl has one or more heteroatoms selected from nitrogen, or oxygen and is optionally further substituted;
R₂ is selected from hydrogen, halogen, C₁₋₁₀ alkyl, C₂₋₁₀ alkenyl, C₂₋₁₀ alkynyl, C₆₋₁₄ aryl, or nitro, wherein C₁₋₁₀ alkyl, C₂₋₁₀ alkenyl, or C₆₋₁₄ aryl, is optionally substituted with one or more C₆₋₁₄ aryl or halogen;
R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH;
m is 1 to 3; and n is 1 to 10.

3. The compound as claimed in claim 1, wherein B, D, E, and F are independently selected from

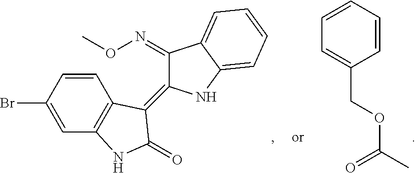 [SMILES: CO/N=C1\C(=C2\C(=O)Nc3cc(Br)ccc32)Nc2ccccc21 ; CC(=O)OCc1ccccc1]

4. The compound as claimed in claim 1, wherein m is 2 and n is 1 to 5.

5. The compound as claimed in claim 1, wherein the compound is of Formula Ia 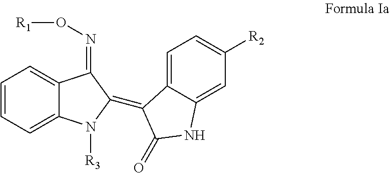
and its polymorphs, stereoisomers, prodrugs, solvates, co-crystals, intermediates, pharmaceutically acceptable salts, and metabolites thereof,
wherein R₁ is selected from hydrogen, C₁₋₁₀ alkyl, or —((CH₂)ₘ—O—)ₙH, wherein C₁₋₁₀ alkyl is optionally substituted with one or more groups selected from C₆₋₁₄ aryl, —B, —COO—C₁₋₁₀ alkyl-D, wherein —COO—C₁₋₁₀ alkyl-D is optionally substituted with —OCO—C₁₋₁₀ alkyl-E optionally substituted with —F, wherein B, D, E and F are independently selected from 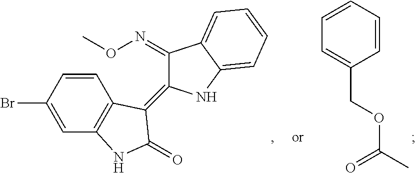 [SMILES: CO/N=C1\C(=C2\C(=O)Nc3cc(Br)ccc32)Nc2ccccc21 ; CC(=O)OCc1ccccc1]
R₂ is selected from halogen, C₁₋₁₀ haloalkyl, C₂₋₁₀ alkenyl, or nitro, wherein C₂₋₁₀ alkenyl is optionally substituted with C₆₋₁₄ aryl; R₃ is selected from hydrogen or —((CH₂)ₘ—O—)ₙH; m is 2; and n is 1 to 5.

6. The compound as claimed in claim 1, wherein the compound is selected from
a. (2Z,3E)-6′-bromo-3-(hydroxyimino)-[2,3′-biindolinylidene]-2′-one lithium salt;
b. (2Z,3E)-3-((benzyloxy)imino)-6′-bromo-[2,3′-biindolinylidene]-2′-one;
c. (2Z,3E)-6′-bromo-3-((2-(2-hydroxyethoxy)ethoxy)imino)-[2,3′-biindolinylidene]-2′-one;
d. (2Z,3E)-6′-bromo-3-((2-(2-(2-hydroxyethoxy)ethoxy)ethoxy)imino)-[2,3′-biindolinylidene]-2′-one;
e. (2Z,3E)-6′-bromo-3-(((14-hydroxy-3,6,9,12-tetraoxatetradecyl)oxy)imino)-[2,3′-biindolinylidene]-2′-one;
f. (2Z,3E)-6′-bromo-1-(2-(2-hydroxyethoxy)ethyl)-3-(hydroxyimino)-[2,3′-biindolinylidene]-2′-one;
g. (2Z,3E)-6′-bromo-1-(2-(2-(2-hydroxyethoxy)ethoxy)ethyl)-3-(hydroxyimino)-[2,3′-biindolinylidene]-2′-one;
h. benzyl 3-((I—((Z′-6′-brom′-2′-oxo-[′,3′-biindolinylidene]-3-ylidene)amino)oxy)-2-((((Z)—((Z′-6′-brom′-2′-oxo-[′,3′-biindolinylidene]-3-ylidene)amino)oxy)methyl)-2-methylpropanoate;
i. 2-((benzyloxy)carbonyl)-2-methylpropane-1,3-diyl bis(3-((((2Z,3E′-6′-brom′-2′-oxo-[′,3′-biindolinylidene]-3-ylidene)amino)oxy)-2-(((((2Z,3E′-6′-brom′-2′-oxo-[′,3′-biindolinylidene]-3-ylidene)amino)oxy)methyl)-2-methylpropanoate);
j. (2Z,3E)-1-(2-(2-(2-hydroxyethoxy)ethoxy)ethyl)-3-(hydroxyimino′-6′-((E)-styryl)-[′,3′-biindolinylidene′-2′-one;
k. (2Z,3E′-6′-bromo-3-((2-(2-(2-hydroxyethoxy)ethoxy)ethoxy)imino)-1-(2-(2-(2-hydroxyethoxy)ethoxy)ethyl)-[′,3′-biindolinylidene′-2′-one;
l. (2Z,3E)-3-(hydroxyimino′-6′-nitro-[′,3′-biindolinylidene′-2′-one lithium salt;
m. (2Z,3E)′ 6′-bromo-3-((2-hydroxyethoxy)imino)-[′,3′-biindolinylidene′-2′-one;
n. (2Z,3E)-1-(2-hydroxyethyl)-3-(hydroxyimino′-6′-bromo-[′,3′-biindolinylidene′-2′-one lithium salt;
o. (2Z,3E)-3-(hydroxyimino′-6′-((E)-styryl)-[′,3′-biindolinylidene′-2′-one; and
p. (2Z,3E)-3-(hydroxyimino′-6′-nitro-[′,3′-biindolinylidene′-2′-one.

7. The compound as claimed in claim 1, wherein the compound of Formula I acts as potent inducer of autophagy.

8. An intermediate compound of Formula IB, wherein the compound is selected from (Z′-6′-bromo-1-(2-(2-(2-hydroxyethoxy)ethoxy)ethyl)-[′,3′-biindolinylidene′-2′,3-dione; and (Z′-6′-bromo-1-(2-(2-hydroxyethoxy)ethyl)-[′,3′-biindolinylidene′-2′,3-dione.

9. A process for preparing the compound of Formula I as claimed in claim 1, wherein the process comprises:
reacting a compound of Formula II with a compound of Formula III in the presence of a solvent and a base to obtain the compound of Formula I, 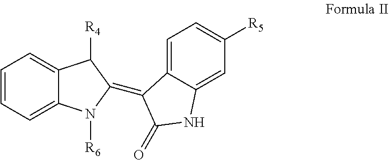
wherein R₄ is selected from hydrogen, ═N—O—H, or ═O;
R₅ is halogen, or nitro;
R₆ is selected from hydrogen, C₁₋₁₀ alkyl, C₂₋₁₀ alkenyl, C₂₋₁₀ alkynyl, or C₆₋₁₄ aryl wherein C₁₋₁₀ alkyl C₂₋₁₀ alkenyl, or C₂₋₁₀ alkynyl is optionally substituted with —(O—(CH₂)ₘ—)ₙ—OH; m is 1 to 3 and n is 1 to 10.

10. The process as claimed in claim 9, wherein the compound of Formula III is selected from A-X, R₂—X, or R₃—X, wherein A is selected from hydrogen, C₁₋₁₀ alkyl, C₂₋₁₀ alkenyl, C₂₋₁₀ alkynyl, C₆₋₁₄ aryl, or —((CH₂)ₘ—O—)ₙH, wherein C₁₋₁₀ alkyl, C₂₋₁₀ alkenyl, C₂₋₁₀ alkynyl, or C₆₋₁₄ aryl is optionally substituted with one or more groups selected from C₁₋₁₀ alkyl, C₂₋₁₀ alkenyl, C₂₋₁₀ alkynyl, C₆₋₁₄ aryl, —B, or —COO—C₁₋₁₀ alkyl-D, wherein —COO—C₁₋₁₀ alkyl-D is optionally substituted with —OCO—C₁₋₁₀ alkyl-E, or —COO—C₁₋₁₀ alkyl-E, wherein —OCO—C₁₋₁₀ alkyl-E, or —COO—C₁₋₁₀ alkyl-E is optionally substituted with —F, wherein B is selected from —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur, and is optionally further substituted; wherein D, E, and F are independently selected from C₁₋₁₀ alkyl, C₂₋₁₀ alkenyl, C₂₋₁₀ alkynyl, C₆₋₁₄ aryl, —COO—CH₂—C₆₋₁₄ aryl, C₁₋₂₀ heterocyclyl, or —O—N—C₁₋₂₀ heterocyclyl, wherein C₁₋₂₀ heterocyclyl, —O—N—C₁₋₂₀ heterocyclyl has one or more heteroatoms selected from nitrogen, oxygen, or sulphur and is further optionally substituted;
R₂ is selected from hydrogen, halogen, C₁₋₁₀ alkyl, C₂₋₁₀ alkenyl, C₂₋₁₀ alkynyl, C₆₋₁₄ aryl, nitro, amine, or amide, wherein C₁₋₁₀ alkyl, C₂₋₁₀ alkenyl, or C₆₋₁₄ aryl is optionally substituted with one or more C₆₋₁₄ aryl or halogen;
R₃ is selected from hydrogen, halogen, C₁₋₁₀ alkyl, C₂₋₁₀ alkenyl, C₂₋₁₀ alkynyl, C₆₋₁₄ aryl, or —((CH₂)ₘ—O—)ₙH; m is 1 to 3; n is 1 to 10; and
X is selected from hydrogen, halogen, or tosyl.

11. The process as claimed in claim 9, wherein B, D, E and F are independently selected from

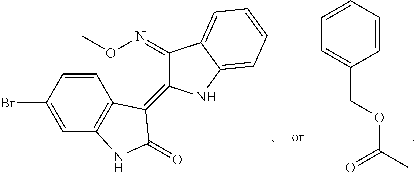 [SMILES: CO/N=C1\C(=C2\C(=O)Nc3cc(Br)ccc32)Nc2ccccc21 ; CC(=O)OCc1ccccc1]

12. The process as claimed in claim 9, wherein the solvent is selected from tetrahydrofuran, pyridine, dimethyl furan, methanol, N,N′-dimethyl formamide, dichloromethane, or combinations thereof; and the base is selected from potassium carbonate, triethyl amine, sodium bicarbonate, pyridine, lithium hydroxide, sodium hydroxide, lithium hydroxide, or combinations thereof.

13. The process as claimed in claim 9, wherein the process comprises a catalyst selected from palladium acetate, tri(o-tolyl)phosphine, or combinations thereof.

14. A pharmaceutical composition comprising a compound of Formula I or a pharmaceutically acceptable salt thereof as claimed in claim 1 together with a pharmaceutically acceptable carrier, optionally in combination with one or more other pharmaceutical compositions.

15. The pharmaceutical composition as claimed in claim 14, wherein the composition is in the form selected from the group consisting of a tablet, capsule, powder, syrup, solution, aerosol, and suspension.

16. The pharmaceutical composition as claimed in claim 14, wherein the composition acts as potent inducer of autophagy.

17. A method for the treatment and/or prevention of various diseases, including intellectual disability (ID) or autism spectrum disorder comprising administering to a subject suffering from functional, behavioral, neurodegenerative disorders or intellectual disability (ID) or autism spectrum disorder a therapeutically effective amount of the compound of Formula I as claimed in claim 1 to a subject in need thereof.

18. A method for the treatment and/or prevention of various diseases, including intellectual disability (ID) or autism spectrum disorder, comprising administering to a subject suffering functional, behavioral, neurodegenerative disorders or intellectual disability (ID) or autism spectrum disorder a therapeutically effective amount of the compound as claimed in claim 1, with other clinically relevant agents to a subject in need thereof.

19. (canceled)
